(19) United States
(12) Patent Application Publication
(10) Pub. No.: US 2023/0016320 A1
(43) Pub. Date: Jan. 19, 2023

(54) IMAGE ANALYSIS METHOD, IMAGE GENERATION METHOD, LEARNING-MODEL GENERATION METHOD, ANNOTATION APPARATUS, AND ANNOTATION PROGRAM

(71) Applicant: Sony Group Corporation, Tokyo (JP)

(72) Inventors: Yuki Ono, Kanagawa (JP); Kazuki Aisaka, Kanagawa (JP); Toya Teramoto, Kanagawa (JP)

(73) Assignee: Sony Group Corporation, Tokyo (JP)

(21) Appl. No.: 17/784,603

(22) PCT Filed: Dec. 18, 2020
(86) PCT No.: PCT/JP2020/047324

§ 371 Date: Jun. 10, 2022

(30) Foreign Application Priority Data
Dec. 19, 2019 (JP) 2019-229048

(51) Int. Cl. G06T 7/00; G06T 11/00; G06T 7/11; G16H 30/40

(52) U.S. Cl. CPC G06T 7/0012; G06T 11/00; G06T 7/11; G16H 30/40; G06T 2207/10056; G06T 2207/30096; G06T 2207/20081; G06T 2207/10068; G06T 2207/10088; G06T 2207/10081; G06T 2207/20016

(57) ABSTRACT

The usability in annotating an image of a subject derived from a living body is improved. An image analysis method is implemented by one or more computers and includes: displaying a first image that is an image of a subject derived from a living body; acquiring information regarding a first region based on a first annotation added to the first image by a user (S101); specifying a similar region similar to the first region from a region different from the first region in the first image, or a second image obtained by image capture of a region including at least a part of a region of the subject subjected to capture of the first image, based on the information regarding the first region (S102, S103); and displaying a second annotation in a second region corresponding to the similar region in the first image (S104).

### FIELD

The present invention relates to an image analysis method, an image generation method, a learning-model generation method, an annotation apparatus, and an annotation program.

### BACKGROUND

In recent years, there has been developed a technique of adding an information tag (meta data, which will be hereinafter referred to as an “annotation”) to a region where a lesion is likely to be present, or the like in an image of a subject derived from a living body, to mark the region as a target region of interest. The annotated image can be used as training data for machine learning. For example, in a case where a target region is a lesion, an image of the target region added with an annotation is used as training data for machine learning, thereby constructing artificial intelligence (AI) that automatically performs diagnosis based on the image. With this technique, improvement in accuracy of diagnosis can be expected.

Meanwhile, in an image including a plurality of target regions of interest, all of the target regions are required to be annotated in some cases. For example, Non-Patent Literature 1 discloses a technique in which a user such as a pathologist traces a lesion or the like in a displayed image using an input device (a mouse, an electronic pen, or the like, for example) to designate a target region. The designated target region is annotated. In this way, the user attempts to annotate all the target regions included in the image.

### CITATION LIST

### Non Patent Literature

Non Patent Literature 1: “Annotation of Whole Slide Images Using Touchscreen Technology”, by Jessica L. Baumann et al., Pathology Visions 2018

### SUMMARY

### Technical Problem

However, in the above-described conventional technique, there is room for further improvement to promote improvement in usability. For example, as a user annotates target regions individually, it takes a huge amount of time and effort to complete the annotation operation.

Thus, the present disclosure has been made in view of the above-described problem, and proposes an image analysis method, an image generation method, a learning-model generation method, an annotation apparatus, and an annotation program capable of improving usability in annotating an image of a subject derived from a living body.

### Solution to Problem

An image analysis method according to an embodiment of the present disclosure is implemented by one or more computers and includes: displaying a first image that is an image of a subject derived from a living body; acquiring information regarding a first region based on a first annotation added to the first image by a user; and specifying a similar region similar to the first region from a region different from the first region in the first image, or a second image obtained by image capture of a region including at least a part of a region of the subject subjected to capture of the first image, based on the information regarding the first region, and displaying a second annotation in a second region corresponding to the similar region in the first image.

### BRIEF DESCRIPTION OF DRAWINGS

FIG. 1 is a view illustrating an image analysis system according to embodiments.

FIG. 2 is a view illustrating a configuration example of an image analyzer according to an embodiment.

FIG. 3 is a view illustrating an example of calculation of the feature value of a target region.

FIG. 4 is a view illustrating an example of a mipmap for explaining a method of acquiring an image being searched.

FIG. 5 includes views illustrating examples of search of a target region based on a user's input.

FIG. 6 includes views illustrating examples of a pathological image for explaining display processing of the image analyzer.

FIG. 7 includes views illustrating examples of search of a target region based on a user's input.

FIG. 8 is a flowchart illustrating a processing procedure according to the embodiment.

FIG. 9 includes views illustrating examples of a pathological image for explaining search processing of the image analyzer.

FIG. 10 includes views illustrating examples of a pathological image for explaining search processing of the image analyzer.

FIG. 11 includes views illustrating examples of a pathological image for explaining search processing of the image analyzer.

FIG. 12 is a view illustrating a configuration example of an image analyzer according to an embodiment.

FIG. 13 includes explanatory views for explaining processing of generating an annotation from the feature value of a super-pixel.

FIG. 14 includes explanatory views for explaining processing of calculating affinity vectors.

FIG. 15 includes explanatory views for explaining processing of denoising a super-pixel.

FIG. 16 is a view illustrating an example of designation of a target region using a super-pixel.

FIG. 17 includes views illustrating examples of a pathological image for explaining visualization in the image analyzer.

FIG. 18 includes views illustrating examples of designation of a target region using a super-pixel.

FIG. 19 is a flowchart illustrating a processing procedure according to the embodiment.

FIG. 20 is a view illustrating an example of detection of cell nuclei.

FIG. 21 includes views illustrating examples of how cell nuclei look.

FIG. 22 is a view illustrating an example of the flatness of normal cell nuclei.

FIG. 23 is a view illustrating an example of the flatness of abnormal cell nuclei.

FIG. 24 is a view illustrating an example of a distribution of the feature values of cell nuclei.

FIG. 25 is a flowchart illustrating a processing procedure according to the embodiment.

FIG. 26 includes views illustrating a success example and a failure example of a super-pixel.

FIG. 27 is a view illustrating an example of a target region based on the success example of a super-pixel.

FIG. 28 is a view illustrating an example of a target region based on the failure example of a super-pixel.

FIG. 29 is a view illustrating an example of generation of a learning model specialized for each organ.

FIG. 30 is a view illustrating an example of a combination of images corresponding to correct-answer information for machine learning.

FIG. 31 is a view illustrating an example of a combination of images corresponding to incorrect-answer information for machine learning.

FIG. 32 is a view illustrating an example in which a target region in a pathological image is displayed in a visually recognizable manner.

FIG. 33 is a view illustrating an example of information processing in learning by machine learning.

FIG. 34 is a hardware configuration diagram illustrating an example of a computer that implements the functions of the image analyzer.

### DESCRIPTION OF EMBODIMENTS

Below, modes (hereinafter referred to as “embodiments”) for implementing an image analysis method, an image generation method, a learning-model generation method, an annotation apparatus, and an annotation program according to the present application will be described in detail with reference to the drawings. The image analysis method, the image generation method, the learning-model generation method, the annotation apparatus, and the annotation program are not limited to the embodiments. In each of the following embodiments, the same parts are denoted by the same reference signs, and duplicated description is omitted.

The present disclosure will be described in the following order of items.

1. Configuration of system according to embodiments

2. First Embodiment

2.1. Image analyzer according to the first embodiment

2.2. Image processing according to the first embodiment

2.3. Processing procedure according to the first embodiment

3. Second Embodiment

3.1. Image analyzer according to the second embodiment

3.2. Image processing according to the second embodiment

3.3. Processing procedure according to the second embodiment

4. Modifications of the second embodiment

4.1. First modification: search using cell information

4.1.1. Image analyzer

4.1.2. Information processing

4.1.3. Processing procedure

4.2. Second modification: search using organ information

4.2.1. Image analyzer

4.2.2. Variations of information processing

4.2.2.1. Acquisition of correct-answer information with a small amount of data for correct-answer information

4.2.2.2. Learning using combination of images corresponding to incorrect-answer information

4.2.2.3. Acquisition of incorrect-answer information with a small amount of data for incorrect-answer information

4.3. Third modification: search using staining information

4.3.1. Image analyzer

5. Application examples of embodiments

6. Other variations

7. Hardware Configuration

8. Other

### Embodiments

### 1. Configuration of System According to Embodiments

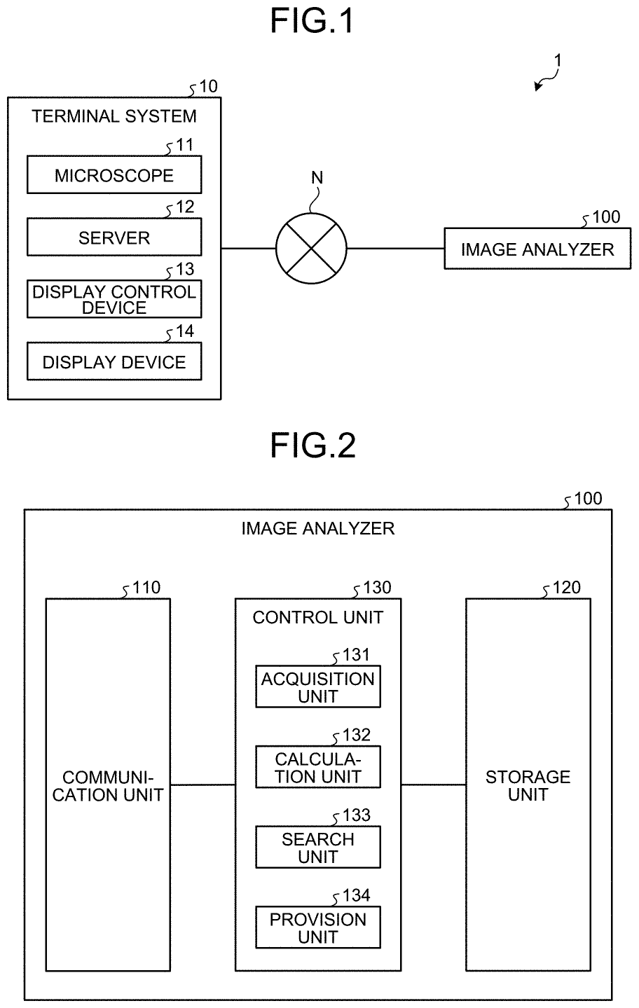

First, an image analysis system 1 according to embodiments will be described with reference to FIG. 1. FIG. 1 is a view illustrating the image analysis system 1 according to the embodiments. As illustrated in FIG. 1, the image analysis system 1 includes a terminal system 10 and an image analyzer 100 (or an image analyzer 200). Additionally, the image analysis system 1 illustrated in FIG. 1 may include a plurality of terminal systems 10 and a plurality of image analyzers 100 (or image analyzers 200).

The terminal system 10 is a system used mainly by a pathologist and is applied to, for example, a laboratory or a hospital. As illustrated in FIG. 1, the terminal system 10 includes a microscope 11, a server 12, a display control device 13, and a display device 14.

The microscope 11 is, for example, an imaging device that images an observed object placed on a glass slide and captures a pathological image (an example of a microscopic image) that is a digital image. An observed object is, for example, a tissue or a cell collected from a patient, and may be a piece of an organ, saliva, blood, or the like. The microscope 11 sends a pathological image as acquired, to the server 12. Additionally, the terminal system 10 is not necessarily required to include the microscope 11. That is, the terminal system 10 is not limited to the configuration that captures a pathological image using the microscope 11 provided therein, and may have a configuration that acquires a pathological image captured by an external imaging device (an imaging device provided in another terminal system, for example) via a predetermined network or the like.

The server 12 is a device that holds a pathological image in a storage area provided therein. A pathological image held by the server 12 can include a pathological image having been subjected to pathological diagnosis by a pathologist, for example. Upon receipt of a viewing request from the display control device 13, the server 12 searches the storage area for a pathological image and transmits the pathological image having been searched for, to the display control device 13.

The display control device 13 transmits a viewing request for a pathological image, received from a user such as a pathologist, to the server 12. Further, the display control device 13 controls the display device 14 to cause the display device 14 to display a pathological image requested by the server 12.

In addition, the display control device 13 accepts a user's operation on a pathological image. The display control device 13 controls the pathological image displayed on the display device 14 in accordance with the accepted operation. For example, the display control device 13 accepts a change in the display magnification of a pathological image. Then, the display control device 13 controls the display device 14 to cause the display device 14 to display a pathological image at the changed display magnification.

Moreover, the display control device 13 accepts an operation of annotating a target region on the display device 14. Then, the display control device 13 transmits the positional information of the annotation added by the operation, to the server 12. As a result, the positional information of the annotation is held in the server 12. Further, upon receipt of a viewing request for the annotation from a user, the display control device 13 transmits the viewing request for the annotation, received from the user, to the server 12. Then, the display control device 13 controls the display device 14 so that the annotation, received from the server 12 is displayed while being superimposed on a pathological image, for example.

The display device 14 includes a screen using liquid crystal, electro-luminescence (EL), a cathode ray tube (CRT), or the like, for example. The display device 14 may be compatible with 4K or 8K, or may include a plurality of display devices. The display device 14 displays a pathological image which is displayed under the control of the display control device 13. A user performs an operation of annotating a pathological image while viewing the pathological image displayed on the display device 14. As described above, a user can annotate a pathological image while viewing the pathological image displayed on the display device 14, which allows the user to freely designate a target region that the user desires to note on the pathological image.

Furthermore, the display device 14 can also display various types of information added to a pathological image. The various types of information include, for example, an annotation added by a user to a pathological image. For example, with display of an annotation superimposed on a pathological image, a user can perform pathological diagnosis based on an annotated target region.

In the meantime, the accuracy of pathological diagnosis varies among pathologists. Specifically, a diagnosis result for a pathological image may vary among pathologists depending on the years of experience, expertise, and the like of each pathologist. For this reason, in recent years, there has been developed a technique of extracting diagnosis aiding information for aiding in pathological diagnosis by machine learning, in order to aid a pathologist or the like in pathological diagnosis. Specifically, there has been proposed a technique in which a plurality of pathological images including annotated target regions of interest are prepared and machine learning is performed using the pathological images as training data, thereby estimating a target region of interest in a new pathological image. According to this technique, a region of interest in a pathological image can be provided to a pathologist, which allows the pathologist to more appropriately perform pathological diagnosis on the pathological image.

However, the practice followed by a pathologist in performing pathological diagnosis is only to observe a pathological image, and the pathologist hardly annotates a region that affects the pathological diagnosis, such as a lesion. Thus, the above-described technique of extracting diagnosis aiding information using machine learning, in which a large amount of learning data is prepared by an operation of annotating a pathological image, requires a lot of time and operators for annotating. If a sufficient amount of learning data cannot be prepared, the accuracy of machine learning decreases, and it becomes difficult to extract diagnosis aiding information (that is, a region of interest in a pathological image) with high accuracy. Additionally, while there is a scheme of weakly-supervised learning that does not require detailed annotation data, this would cause a problem of lower accuracy than that of machine learning using detailed annotation data.

Thus, in the following embodiments, there will be proposed an image analysis method, an image generation method, a learning-model generation method, an annotation apparatus, and an annotation program capable of improving usability in annotating an image of a subject derived from a living body. For example, the image analyzer 100 (or the image analyzer 200) of the image analysis system 1 according to the embodiments calculates the feature value of a target region designated by a user on a pathological image to specify another target region similar to the target region, and annotates another target region.

### 2. First Embodiment

### 2-1. Image Analyzer According to First Embodiment

Next, the image analyzer 100 according to a first embodiment will be described with reference to FIG. 2. FIG. 2 is a view illustrating an example of the image analyzer 100 according to the embodiment. As illustrated in FIG. 2, the image analyzer 100 is a computer including a communication unit 110, a storage unit 120, and a control unit 130.

The communication unit 110 is realized by a network interface card (NIC) or the like, for example. The communication unit 110 is connected to a network N (not illustrated) over wires or wirelessly, and transmits and receives information to and from the terminal system 10 and the like via the network N. The control unit 130 described later transmits and receives information to and from those devices via the communication unit 110.

The storage unit 120 is realized by, for example, a semiconductor memory element such as a random access memory (RAM) or a flash memory, or a storage device such as a hard disk or an optical disk. The storage unit 120 stores therein information regarding another target region searched for by the control unit 130. Information regarding another target region will be described later.

Further, the storage unit 120 stores therein an image of a subject, an annotation added by a user, and an annotation added to another target region while bringing them into correspondence with each other. On the other hand, the control unit 130 generates an image for generating a learning model (an example of a discriminant function) based on the information stored in the storage unit 120, for example. For example, the control unit 130 generates one or more partial images for generating a learning model. Then, the control unit 130 generates a learning model based on the one or more partial images.

The control unit 130 is realized by execution of a program (an example of an image analysis program) stored in the image analyzer 100 using a RAM or the like as a work area in a central processing unit (CPU) or a microprocessing unit (MPU), for example. However, the control unit 130 is not limited thereto and may be realized by an integrated circuit such as an application specific integrated circuit (ASIC) or a field programmable gate array (FPGA), for example.

As illustrated in FIG. 2, the control unit 130 includes an acquisition unit 131, a calculation unit 132, a search unit 133, and a provision unit 134, and implements or performs functions or operations for information processing described below. Additionally, the internal configuration of the control unit 130 is not limited to the configuration illustrated in FIG. 2, and may be any configuration that can perform the information processing described later.

The acquisition unit 131 acquires a pathological image via the communication unit 110. Specifically, the acquisition unit 131 acquires a pathological image stored in the server 12 of the terminal system 10. Further, the acquisition unit 131 acquires the positional information of an annotation corresponding to a target region designated by a user's input of a boundary to a pathological image displayed on the display device 14, via the communication unit 110. Hereinafter, the positional information of an annotation corresponding to a target region is referred to as “positional information of a target region” if appropriate.

Furthermore, the acquisition unit 131 acquires information regarding the target region based on the annotation added to the pathological image by the user. However, the operation of the acquisition unit 131 is not limited thereto and the acquisition unit 131 may acquire information regarding the target region based on a new annotation generated based on the annotation added by the user, a corrected annotation, or the like (which will be hereinafter collectively referred to as a new annotation). For example, the acquisition unit 131 may acquire information regarding the target region corresponding to a new annotation generated by correction of the annotation added by the user along the contour of a cell. Additionally, generation of the new annotation may be achieved in the acquisition unit 131 or may be achieved in another unit such as the calculation unit 132. Furthermore, as a method of correcting an annotation along the contour of a cell, correction using adsorption fitting or the like may be used, for example. Additionally, adsorption fitting may be, for example, processing in which a curve drawn by a user on a pathological image is corrected (fitted) so as to overlap the contour of a target region having a contour most similar to the curve. However, the generation of the new annotation is not limited to the attraction fitting described above as an example. Various methods such as a method of generating an annotation having a randomly-selected shape (a rectangle or a circle, for example) from the annotation added by the user, may be used, for example.

The calculation unit 132 calculates the feature value of an image included in a target region based on a pathological image acquired by the acquisition unit 131 and the positional information of the target region.

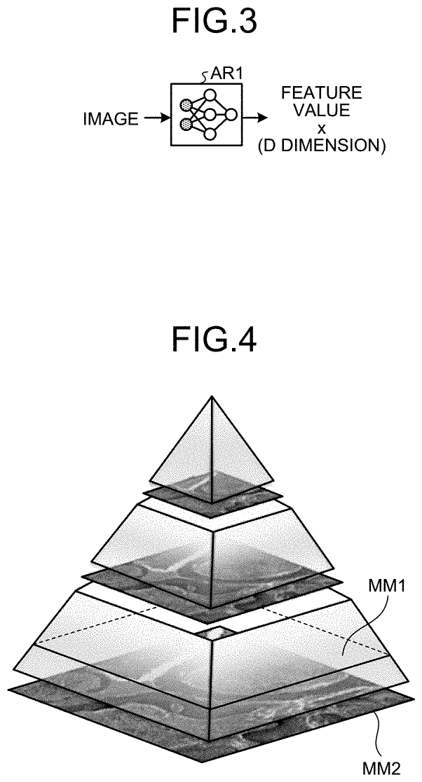

FIG. 3 is a view illustrating an example of calculation of the feature value of a target region. As illustrated in FIG. 3, the calculation unit 132 inputs an image included in a target region to an algorithm AR1 such as a neural network to calculate the feature value of the image. In FIG. 3, the calculation unit 132 calculates the feature value of the image in each D-dimension indicating the feature of the image. Then, the calculation unit 132 calculates a representative feature value that is a feature value of the whole of a plurality of target regions by aggregating the respective feature values of the images included in the plurality of target regions. For example, the calculation unit 132 calculates a representative feature value of the whole of a plurality of target regions based on the feature value such as a distribution (a color histogram, for example) of the feature values of the images included in the plurality of target regions or local binary patterns (LBP) focusing on the texture structures of the images. In another example, the calculation unit 132 generates a learning model by learning a representative feature value of the whole of a plurality of target regions using deep learning such as convolutional neural network (CNN). Specifically, the calculation unit 132 generates a learning model by learning in which an image of the whole of a plurality of target regions is input information and a representative feature value of the whole of the plurality of target regions is output information. Then, the calculation unit 132 inputs the image of the whole of the plurality of target regions of interest, to the generated learning model, to calculate the representative feature value of the whole of the plurality of target regions of interest.

Based on the feature value of a target region calculated by the calculation unit 132, the search unit 133 searches for another target region similar to the target region among regions included in a pathological image. The search unit 133 searches for another target region similar to a target region in a pathological image, based on similarly between the feature value of the target region calculated by the calculation unit 132 and the feature value of a region other than the target region in the pathological image. For example, the search unit 133 searches a pathological image of a subject or another pathological image obtained by image capture of a region including at least a part of the pathological image, for another target region similar to a target region. The similar region may be extracted from a predetermined region of the pathological image of the subject or the image obtained by image capture of the region including at least a part of the pathological image, for example. The predetermined region may be, for example, the whole of an image, a display area, or a region set by a user in the image. Further, the same angle of view may include an angle of view of images having different focal points, such as a Z stack.

In an example of a method of acquiring an image being searched by the search unit 133, the image analyzer 100 may acquire an original image of a region of interest (ROI) from the closest layer at a higher magnification than the display magnification of the screen, for example. In addition, depending on the type of lesion, it may be desired to view a wide range in one case and it may be desired to enlarge a specific cell in another case, in order to check the spread of the lesion. As such, required resolution may differ according to cases. In such a case, the image analyzer 100 may acquire an image with appropriate resolution depending on the type of lesion. FIG. 4 illustrates an example of a mipmap for explaining a method of acquiring an image being searched. As illustrated in FIG. 4, the mipmap has a pyramidal hierarchical structure in which a lower layer has a higher magnification (also referred to as resolution). Respective layers are whole-slide images having different magnifications. A layer MM1 is a layer at the display magnification of the screen, and a layer MM2 is a layer at the acquisition magnification of an image acquired by the image analyzer 100 for search processing by the search unit 133. Thus, the layer MM2 lower than the layer MM1 is a layer having a higher magnification than that of the layer MM1, and may be, for example, a layer at the highest magnification. The image analyzer 100 acquires an image from the layer MM2. By using the mipmap having such a hierarchical structure, the image analyzer 100 can perform processing without image degradation.

Additionally, it is possible to generate the mipmap having the above-described hierarchical structure, for example, by photographing a subject with high resolution and gradually reducing the resolution of the high-resolution image of the whole of the subject obtained by the photographing to generate image data of each layer. More specifically, first, a subject is photographed a plurality of times with high resolution. The plurality of high-resolution images thus obtained are joined together by stitching, to be converted into one high-resolution image (corresponding to a whole-slide image) that shows the whole of the subject. Additionally, the high-resolution image corresponds to the lowermost layer in the pyramidal structure of the mipmap. Subsequently, the high-resolution image showing the whole of the subject is divided into a plurality of images of the same size in a grid form (hereinafter referred to as tile images). Then, the predetermined number of tile images of M×N (M and N are integers of two or more) are subjected to down sampling to perform processing of generating one tile image of the same size on the whole of the current layer, thereby generating an image of a layer higher than the current layer by one. The image generated in this manner is an image showing the whole of the subject and is divided into a plurality of tile images. Thus, by repetition of the above-described down sampling for each layer up to the uppermost layer, a mipmap having a hierarchical structure can be generated. However, the method is not limited to the above-described generation method, and any of various methods that can generate a mipmap with layers having different resolutions, can be used.

When the image analyzer 100 acquires information regarding an image of a subject displayed at the time of a user's annotating (including information such as resolution, a magnification, or a layer, for example, and hereinafter referred to as “image information”), the image analyzer 100 acquires an image at a magnification equal to or higher than the magnification specified from the image information, for example. Then, the image analyzer 100 determines an image that is to be searched for a similar region, based on the acquired image information. Additionally, the image analyzer 100 may select an image that is to be searched for a similar region, among images having the same resolution, lower resolution, and higher resolution as compared to the resolution specified from the image information, according to a purpose. Moreover, while this description deals with a case where an image being searched is acquired based on resolution as an example, the image information is not limited to resolution. The acquisition of the image may be based on various types of information such as a magnification and a layer.

Furthermore, the image analyzer 100 acquires images with different resolutions from the images stored in the pyramidal hierarchical structure. For example, the image analyzer 100 acquires, for example, an image having higher resolution than that of an image of a subject displayed at the time of a user's annotating. In this case, the image analyzer 100 may reduce and display the acquired high-resolution image to a size corresponding to the magnification specified by the user (corresponding to the magnification of the image of the subject displayed at the time of the user's annotating), for example. For example, the image analyzer 100 may reduce and display an image having the lowest resolution among images having resolution higher than the resolution corresponding to the magnification specified by the user. In this manner, by designating a similar region among images having resolution higher than that of the image of the subject displayed at the time of the user's annotating, it is possible to improve the search accuracy of the similar region.

Additionally, in a case where there is no image having higher resolution than that of an image of a subject displayed at the time of a user's annotating, for example, the image analyzer 100 may acquire an image having the same resolution as that of the image of the subject.

Furthermore, for example, the image analyzer 100 may specify resolution suitable for similar-region search based on the state of a subject specified from an image of the subject, a diagnosis result, or the like, and acquire an image with the specified resolution. Resolution required for generating a learning model differs depending on the state of a subject such as the type or the progression stage of a lesion. Then, with the above-described configuration, it is possible to generate a more accurate learning model according to the state of the subject.

Further, the image analyzer 100 may acquire, for example, an image having lower resolution than that of an image of a subject displayed at the time of a user's annotating, for example. In this case, the amount of data being processed can be reduced, which can shorten a time required for search, learning, and the like of a similar region.

Moreover, the image analyzer 100 may acquire images in different layers of the pyramidal hierarchical structure and generate an image of a subject or an image being searched, from the acquired images. For example, the image analyzer 100 may generate an image of a subject from an image having higher resolution than that of the image. Furthermore, for example, the image analyzer 100 may generate an image being searched from an image having higher resolution than that of the image.

The provision unit 134 provides the positional information of another target region searched for by the search unit 133, to the display control device 13. Upon receipt of the positional information of another target region from the provision unit 134, the display control device 13 controls a pathological image so that another target region is annotated. The display control device 13 controls the display device 14 to cause the display device 14 to display the annotation added to another target region.

### 2-2. Image Processing According to First Embodiment

While the acquisition unit 131 acquires the positional information of a target region as described above, the positional information of the target region acquired by the acquisition unit 131 depends on a method in which a user inputs a boundary on a pathological image. There are two methods for a user to input a boundary. These two methods are a method of inputting (stroking) a boundary to the entire contour of a living body, and a method of filling in the contour of a living body to input a boundary. In both methods, a target region is designated based on the input boundary.

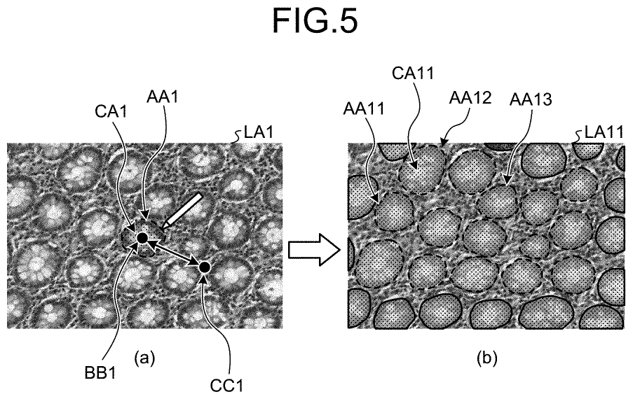

FIG. 5 illustrates pathological images showing living bodies such as cells. A method in which the acquisition unit 131 acquires the positional information of a target region designated by a user's input of a boundary to the entire contour of a living body will be described with reference to FIG. 5. In FIG. 5(a), a user inputs a boundary to the entire contour of a living body CA1 included in a pathological image. In FIG. 5(a), when the user inputs the boundary, an annotation AA1 is added to the living body CA1 to which the boundary has been input. In a case where the user inputs a boundary to the entire contour of the living body CA1 as illustrated in FIG. 5(a), the annotation AA1 is added to the whole of a region surrounded by the boundary. The region indicated by the annotation AA1 is a target region. That is, the target region includes not only the boundary input by the user but also the whole of the region surrounded by the boundary. In FIG. 5(a), the acquisition unit 131 acquires the positional information of the target region. The calculation unit 132 calculates the feature value of the region indicated by the annotation AA1. Based on the feature value calculated by the calculation unit 132, the search unit 133 searches for another target region similar to the target region indicated by the annotation AA1. Specifically, for example, the search unit 133 may use the feature value of the region indicated by the annotation AA1 as a reference and search for a target region having a feature value equal to or more than, or equal to or less than, a predetermined threshold value with respect to the reference, as another target region that is similar. FIG. 5(b) illustrates a search result of another target region similar to the target region designated by the user.

According to this method, the search unit 133 searches for another target region based on comparison (a difference or a ratio, for example) between the feature value of a region inside the target region and the feature value of a region outside the target region. In FIG. 5(b), the search unit 133 searches for another target region based on comparison between the feature value of a randomly-selected region BB1 inside the target region indicated by the annotation AA1 and the feature value of a randomly-selected region CC1 outside the target region indicated by the annotation AA1. The annotations AA11 to AA13 are annotations displayed on the display device 14 based on the positional information of another target region that is searched for by the search unit 133. Additionally, in FIG. 5(b), for the purpose of simplifying the illustration, only the regions indicating the living body CA11 are denoted by reference signs indicating the annotations. Though not all the regions indicating the living body are denoted by reference signs indicating annotations in FIG. 5(b), actually, all the regions having contours indicated by the dotted lines are annotated.

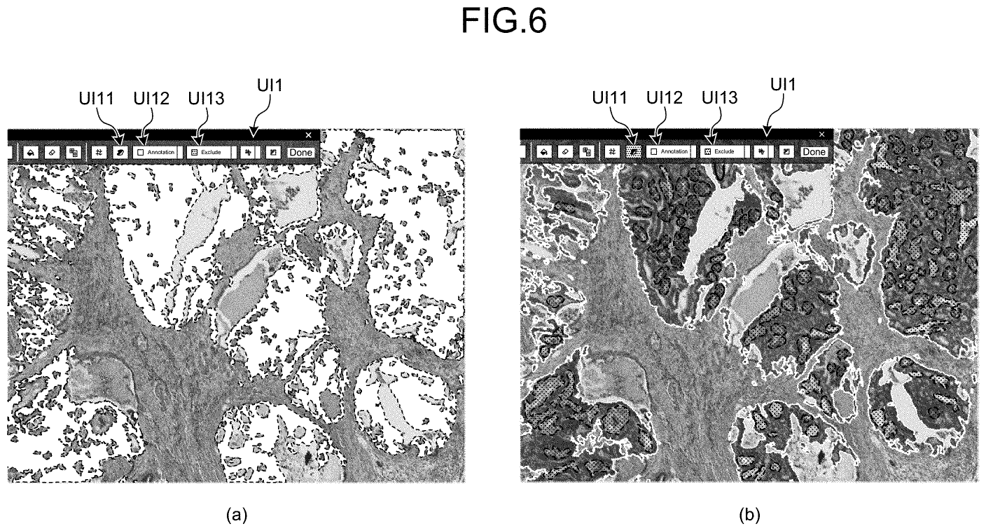

Now, display of an annotated region will be described in detail. The image analyzer 100 switches a display method of an annotated region according to a user's preference, for example. For example, the image analyzer 100 fills in a similar region (estimated region) extracted as another target region similar to a target region with a color designated by a user, and displays it. Below, display processing of the image analyzer 100 will be described with reference to FIG. 6.

FIG. 6 illustrates examples of a pathological image for explaining display processing of the image analyzer 100. FIG. 6(a) illustrates display in a case where a similar region is filled in. When a user selects a display menu UI11 included in a display menu UI1, the screen transitions to the screen of FIG. 6(a). Further, in FIG. 6(a), when the user selects the display menu UI11 included in the display menu UI1, a similar region is filled in with a color designated by a display menu UI12.

The method of displaying an annotated region is not limited to the example of FIG. 6(a). For example, the image analyzer 100 may fill in a region other than a similar region with a color designated by a user and display it

FIG. 6(b) illustrates display in a case where a contour of a similar region is drawn. Description similar to that for FIG. 6(a) is omitted as appropriate. The image analyzer 100 draws the contour of a similar region with a color designated by a user and display it. In FIG. 6(b), when the user selects the display menu UI11 included in the display menu UI1, the contour of a similar region is drawn with a color designated by the display menu UI12. Further, in FIG. 6(b), the contour of a deleted region inside the similar region is drawn. The image analyzer 100 draws the contour of the deleted region inside the similar region with a color designated by the user and display it. In FIG. 6(b), when the user selects the display menu UI11 included in the display menu UI1, the contour of the deleted region inside the similar region is drawn with a color designated by a display menu UI13.

In FIG. 6(b), when the user selects the display menu UI11 included in the display menu UI1, the screen transitions to the screen of FIG. 6(b). Specifically, the screen transitions to the screen in which each of the contours of the similar region and the deleted region inside the similar region is drawn with a color designated by the display menu UI12 or the display menu UI13.

As described above, in FIG. 6, the display method can be switched according to a user's preference, and thus the user can freely select the display method having high visibility according to the user's preference.

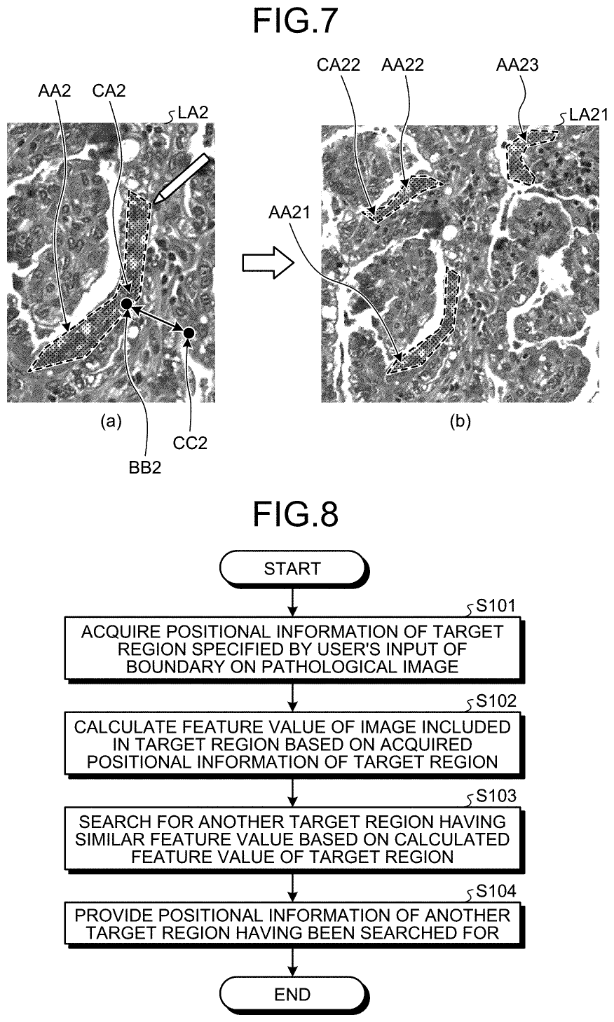

With reference to FIG. 7. a method in which the acquisition unit 131 acquires the positional information of a target region designated by a user's operation of filling in the contour of a living body will be described. In FIG. 7(a), the contour of a living body CA2 included in a pathological image is filled in. In FIG. 7(a), when the user inputs a boundary, an annotation AA22 is added to the contour of the living body CA2 that is filled in by the input of the boundary. The annotation is a target region. As illustrated in FIG. 7(a), when the contour of the living body CA2 is filled in, the annotation AA22 is added to the filled-in region. In FIG. 7(a), the acquisition unit 131 acquires the positional information of the target region. FIG. 7(b) illustrates a search result of another target region similar to the target region designated by the user. In FIG. 7(b), the search unit 133 searches for another target region similar to the target region based on the feature value of the target region indicated by the annotation AA22.

According to this method, the search unit 133 searches for another target region based on comparison between the feature value of a region inside a boundary within which the target region is filled in and the feature value of a region outside the boundary within which the target region is filled in. In FIG. 7(b), the search unit 133 searches for another target region based on comparison between the feature value of a randomly-selected region BB2 inside the boundary within which the target region indicated by the annotation AA22 is filled in and the feature value of a randomly-selected region CC2 outside the boundary within which the target region indicated by the annotation AA22 is filled in. The annotations AA21 to AA23 are annotations displayed on the display device 14 based on the positional information of another target region having been searched for by the search unit 133.

### 2-3. Processing Procedure According to First Embodiment

Next, a processing procedure according to the first embodiment will be described with reference to FIG. 8. FIG. 8 is a flowchart illustrating a processing procedure according to the first embodiment. As illustrated in FIG. 8, the image analyzer 100 acquires the positional information of a target region designated by a user's input of a boundary on a pathological image (step S101).

Further, the image analyzer 100 calculates the feature value of an image included in the target region based on the acquired positional information of the target region (step S102). Subsequently, the image analyzer 100 searches for another target region having a similar feature value based on the calculated feature value of the target region (step S103). Then, the image analyzer 100 provides the positional information of another target region having been searched for (step S104).

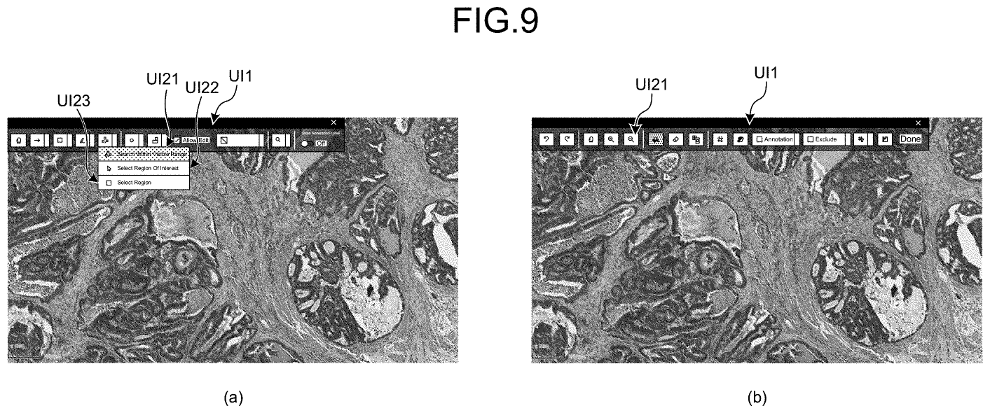

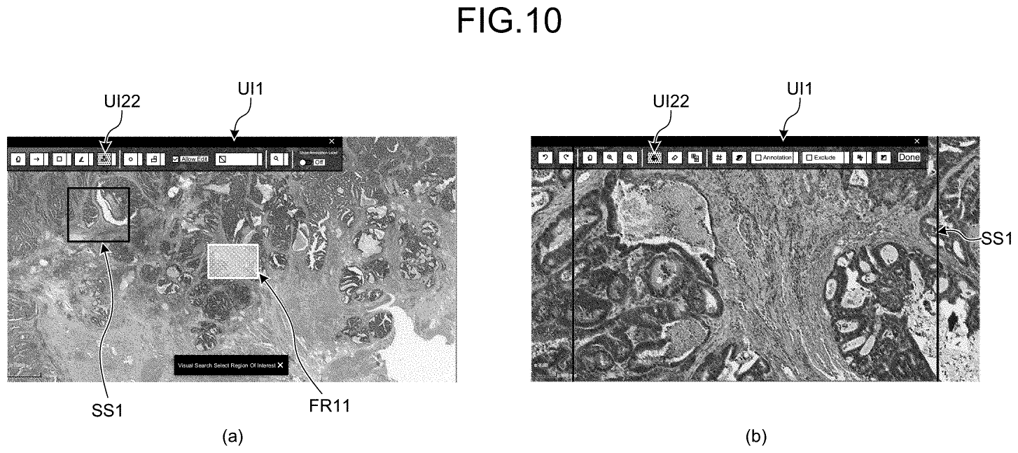

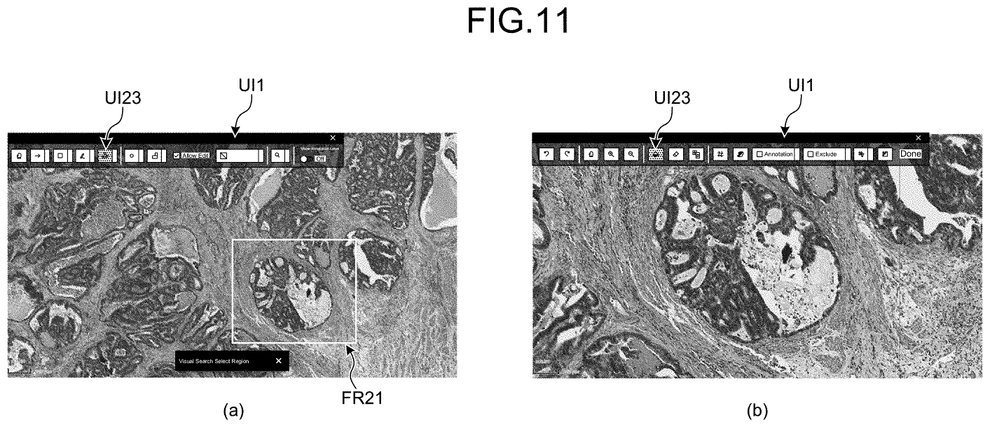

Now, details of the processing in the step S103 will be given. The image analyzer 100 searches at least one of a display area displayed on the display device 14, a first annotated region to which an annotation has been added in advance, and a second annotated region to which an annotation has been newly added, for another target region having a similar feature value. Additionally, those regions are mere examples. The search range is not limited to the three regions, and can be set to any range in which another target region that is similar can be searched for. Further, the image analyzer 100 can have any configuration that can set a range in which another target region that is similar is searched for. Below, search processing of the image analyzer 100 will be described with reference to FIGS. 9 to 11. Additionally, while FIGS. 10 and 11 illustrate a case where the first annotated region and the second annotated region are each displayed in a rectangular shape, the shapes of the regions when displayed are not limited to any particular shape.

FIG. 9 illustrates examples of a pathological image for explaining search processing of the image analyzer 100. Specifically, FIG. 9 illustrates transition of a screen in a case where the image analyzer 100 searches a display area displayed on the display device 14 for another target region having a similar feature value. FIG. 9(a) illustrates a screen before the start of the search processing. FIG. 9(b) illustrates a screen at the start of the search processing. In FIG. 9, when a user selects a display menu UI21 included in the display menu UI1, the screen transitions from FIG. 9(a) to FIG. 9(b). That is, the display menu UI21 is a UI for controlling the image analyzer 100 to cause the image analyzer 100 to perform the search processing on the display area remaining as it is.

Additionally, when the user selects a display menu UI22 included in the display menu U11, a region SS1 (see FIG. 10(a)) that moves to the center of the screen in accordance with the movement of the user's operation through zooming is displayed. Further, when the user selects a display menu UI23 included in the display menu UI11, drawing information (not illustrated) for the user to freely draw the second annotated region is displayed. Additionally, an example of the second annotated region after the drawing is displayed in FIG. 11(a) referred to later.

FIG. 10 illustrates examples of a pathological image for explaining search processing of the image analyzer 100. Specifically, FIG. 10 illustrates transition of a screen in a case where the image analyzer 100 searches the first annotated region for another target region having a similar feature value. FIG. 10(a) illustrates a screen before the start of the search processing. FIG. 10(b) illustrates a screen at the start of the search processing. Additionally, it is assumed that a first annotated region FR21 is displayed in FIG. 10(a).

In FIG. 10, when a user selects the first annotated region FR11, the screen transitions from FIG. 10(a) to FIG. 10(b). For example, when the user performs a mouse-over on the first annotated region FR11 and performs an operation (a click or a tap, for example) on the first annotated region FR11 being highlighted, the screen transitions to FIG. 10(b). Then, in FIG. 10(b), a screen that is zoomed so that the first annotated region FR11 is located at the center is displayed.

FIG. 10 illustrates, for example, a case where a student annotates a ROI having been selected by a pathologist in advance, as an application example.

FIG. 11 illustrates examples of a pathological image for explaining search processing of the image analyzer 100. Specifically, FIG. 11 illustrates transition of a screen in a case where the image analyzer 100 searches the second annotated region for another target region having a similar feature value. FIG. 11(a) illustrates a screen before the start of the search processing. FIG. 11(b) illustrates a screen at the start of the search processing. Additionally, it is assumed that a second annotated region FR21 is displayed in FIG. 11(a).

In FIG. 11, when a user draws the second annotated region FR21, the screen transitions from FIG. 11(a) to FIG. 11(b). Then, in FIG. 11(b), a screen that is zoomed so that the second annotated region FR21 is located at the center is displayed.

FIG. 11 illustrates, for example, a case where a student selects a ROI by himself and annotates it, as an application example.

### 3. Second Embodiment

### 3-1. Image Analyzer According to Second Embodiment

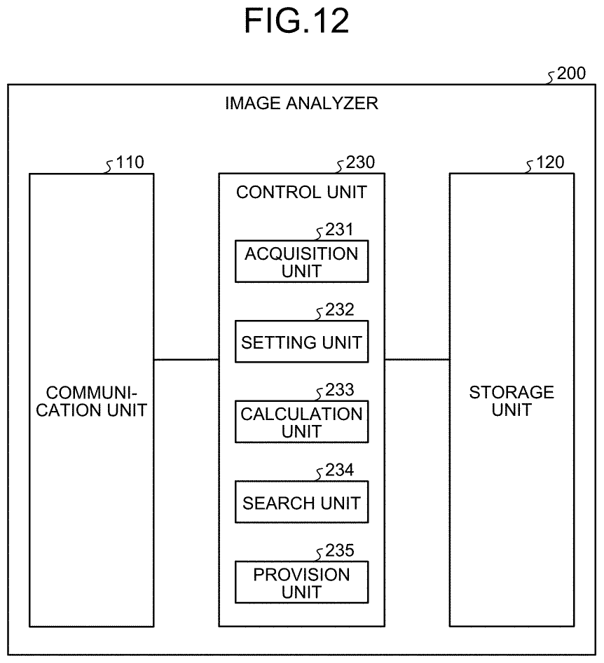

Next, an image analyzer 200 according to a second embodiment will be described with reference to FIG. 12. FIG. 12 is a view illustrating an example of the image analyzer 200 according to the second embodiment. As illustrated in FIG. 12, the image analyzer 200 is a computer including the communication unit 110, the storage unit 120, and a control unit 230. Description similar to that in the first embodiment is omitted as appropriate.

As illustrated in FIG. 12, the control unit 230 includes an acquisition unit 231, a setting unit 232, a calculation unit 233, a search unit 234, and a provision unit 235, and implements or performs functions or operations for information processing described below. Additionally, the internal configuration of the control unit 230 is not limited to the configuration illustrated in FIG. 12, and may be any configuration that can perform information processing described later. Description similar to that in the first embodiment is omitted as appropriate.

The acquisition unit 231 acquires the positional information of a target region designated by a user's selection of a partial region included in a pathological image displayed on the display device 14, via the communication unit 110. Hereinafter, each of partial regions resulted from segmentation based on a feature value of a pathological image will be referred to as a “super-pixel” if appropriate.

The setting unit 232 performs processing of setting a super-pixel in a pathological image. Specifically, the setting unit 232 sets a super-pixel in a pathological image based on segmentation in accordance with the similarity of feature values. More specifically, the setting unit 232 sets a super-pixel in a pathological image by performing segmentation in which pixels with feature values having high similarity are included in the same super-pixel while conforming to the number of segments previously determined by the user.

Furthermore, information regarding the super-pixel set by the setting unit 232 is provided to the display control device 13 by the provision unit 235 described later. Upon receipt of the information regarding the super-pixel provided by the provision unit 235, the display control device 13 controls the display device 14 to cause the display device 14 to display the pathological image in which the super-pixel is set.

The calculation unit 233 calculates the feature value of a target region designated by the user's selection of the super-pixel set by the setting unit 232. Additionally, in a case where a plurality of super-pixels are designated, the calculation unit 233 calculates a representative feature value from the feature values of the super-pixels.

Based on the feature value of the target region calculated by the calculation unit 233, the search unit 234 searches for another target region similar to the target region based on the super-pixel. The search unit 234 searches for another target region similar to the target region, based on similarly between the feature value of the target region calculated by the calculation unit 233 and the feature value of a region that is other than the target region in the pathological image and is based on the super-pixel.

The provision unit 235 provides the positional information of another target region that is searched for by the search unit 234 and is based on the super-pixel, to the display control device 13. Upon receipt of the positional information of another target region from the provision unit 235, the display control device 13 controls the pathological image so that another target region based on the super-pixel is annotated. Below, processing of generating an annotation based on a super-pixel will be described.

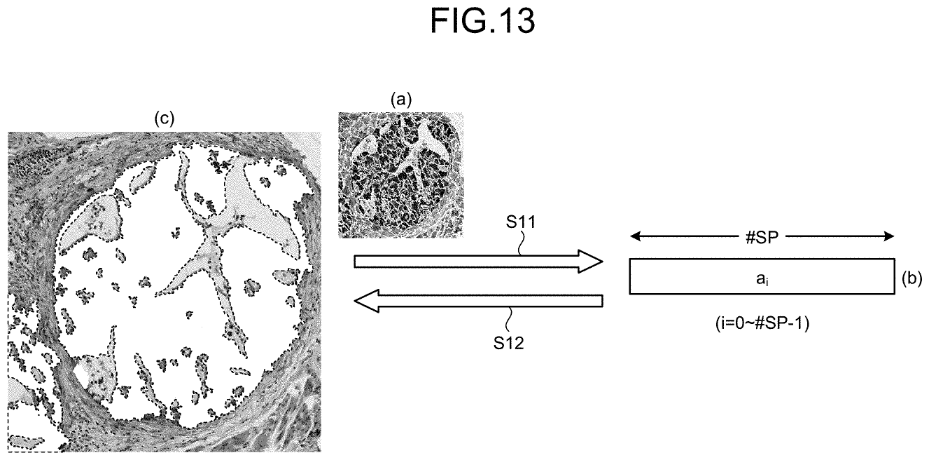

FIG. 13 includes explanatory views for explaining processing of generating an annotation from the feature value of a super-pixel. FIG. 13(a) illustrates a pathological image including a super-pixel. FIG. 13(b) illustrates an affinity vector (aᵢ) that holds similarity between the super-pixel and an annotation being generated (hereinafter referred to as “annotated object” as appropriate). FIG. 13(c) illustrates a pathological image displayed in a case where the similarity of each super-pixel is equal to or greater than a predetermined threshold value. In FIG. 13(b), the length of the affinity vector (aᵢ) is indicated by “#SP”. The number of “#SP” is not limited to any particular number. The image analyzer 100 holds the similarity of each super-pixel to the annotated object (S11). Then, when the similarity of each super-pixel is, for example, is equal to or greater than the predetermined threshold value set in advance, the image analyzer 100 displays all the pixels in the region as an annotated object, to the user (S12). In FIG. 13, the image analyzer 100 displays all of about 10⁶ pixels included in a region having a size of about 10³ to the user as an annotated object.

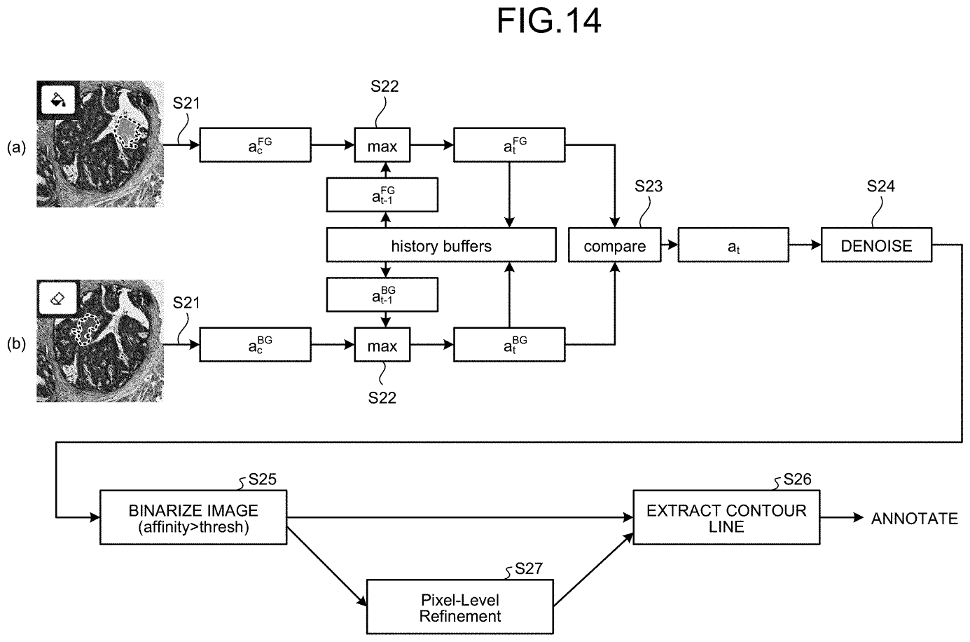

FIG. 14 includes explanatory views for explaining processing of calculating an affinity vector. FIG. 14(a) illustrates addition of an annotation (annotated object). FIG. 14(b) illustrates deletion of an annotation (deleted region). The image analyzer 100 holds the similarity of each of the annotated object and the deleted region to a region input by a user (S21). In FIG. 14, the similarity to the region input by the user is held using class-aware affinity vectors. Here, a_{c}^{FG} indicates a class-aware affinity vector for an annotated object. Further, a_{c}^{BG} indicates a class-aware affinity vector for a deleted region. Additionally, a_{c}^{FG} and a_{c}^{BG} are class-aware affinity vectors based on a region currently input by a user. Further, aₜ₋₁^{FG} and aₜ₋₁^{BG} are class-aware affinity vectors based on a region having been input by a user in the past. The image analyzer 100 specifies a maximum value of a class-aware affinity vector for each of an annotated object and a deleted region (S22). Specifically, the image analyzer 100 calculates a maximum value in consideration of a region input by a user and the input history. For example, the image analyzer 100 calculates a maximum value of an annotated object from a_{c}^{FG} and aₜ₋₁^{FG}. Further, for example, the image analyzer 100 calculates a maximum value of a deleted region from a_{c}^{BG} and aₜ₋₁^{BG}. Then, the image analyzer 100 calculates an affinity vector (aₜ) by comparing a_{c}^{FG} and a_{c}^{BG} (S23).

Thereafter, the image analyzer 100 performs processing of denoising the super-pixel (S24), processing of binarizing the image (S25), and processing of extracting the contour line (S26) to generate an annotation. Additionally, the image analyzer 100 may perform the processing of the step S26 after performing refinement processing (S27) in units of pixels, if necessary, after the step S25.

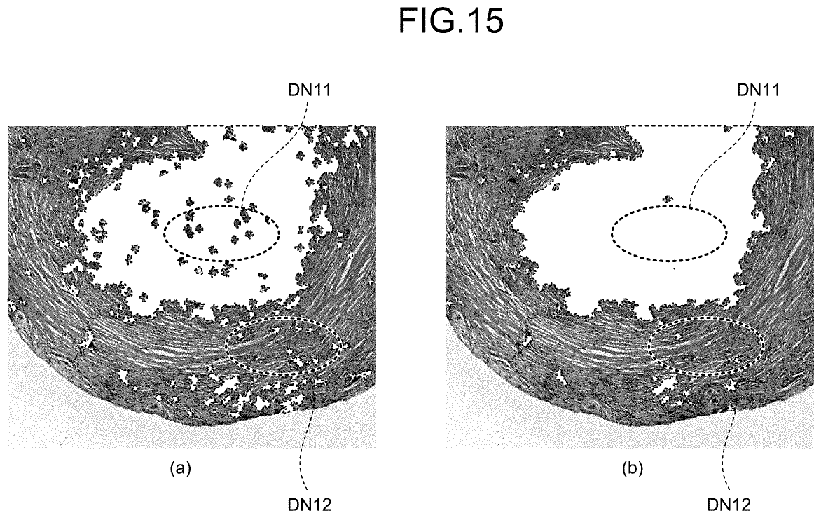

FIG. 15 includes explanatory views for explaining processing of denoising a super-pixel. In some cases, an affinity vector may cause noise-like erroneous detection, un-detection, or the like because it is calculated independently for each super-pixel. In such a case, the image analyzer 100 denoises in consideration of also an adjacent super-pixel, thereby generating a higher-quality output image. FIG. 15(a) illustrates an output image without denoising. In FIG. 15(a), the similarity is calculated independently for each super-pixel, so that noises are displayed in regions indicated by the dotted lines (DN11 and DN12). On the other hand, FIG. 15(b) illustrates an output image having been subjected to denoising. In FIG. 15(b), noises displayed in the regions indicated by the dotted lines in FIG. 15(a) are reduced by denoising.

### 3-2. Image Processing According to Second Embodiment

FIG. 16 illustrates an example of a pathological image in which super-pixels are set. In FIG. 16, a user traces the pathological image to designate the range of super-pixels for calculating a feature value. In FIG. 16, the user designates the range of super-pixels indicated by a region TR11. Additionally, all of the regions surrounded by the white lines are super-pixels. For example, regions TR1 and TR2 surrounded by the dotted lines are examples of a super-pixel. The super-pixels are not limited to the regions TR1 and TR2, and each of all the regions surrounded by the white lines is a super-pixel. In FIG. 16, the feature value of each super-pixel included in the region TR11 is, for example, a feature value SP1 that is the feature value of the region TR1 included in the region TR11 or a feature value SP2 that is the feature value of the region TR2. Though all the super-pixels included in the region TR11 are not denoted by reference signs in FIG. 16 for the purpose of simplifying the illustration, the calculation unit 233 calculates the feature values of all the super-pixels included in the region TR11 in the same manner. The calculation unit 233 calculates the feature value of each super-pixel included in the region TR11 and aggregates the feature values of all the super-pixels to calculate a representative feature value of the range of super-pixels indicated by the region TR11. For example, the calculation unit 233 calculates the average feature value of all the super-pixels included in the region TR11 as a representative feature value. Though all the super-pixels included in the region TR11 are not denoted by reference signs in FIG. 16, the feature values of all the super-pixels included in the region TR11 are calculated in the same manner.

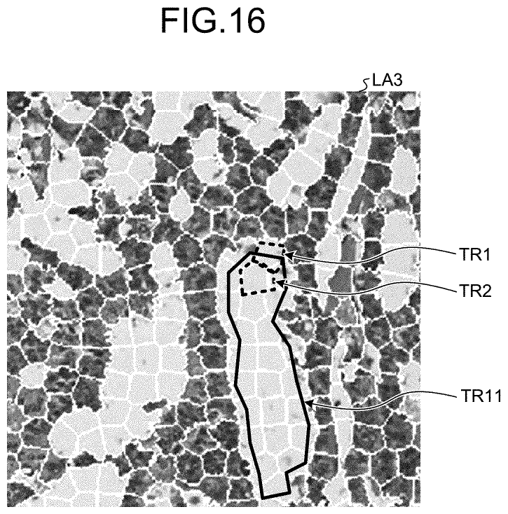

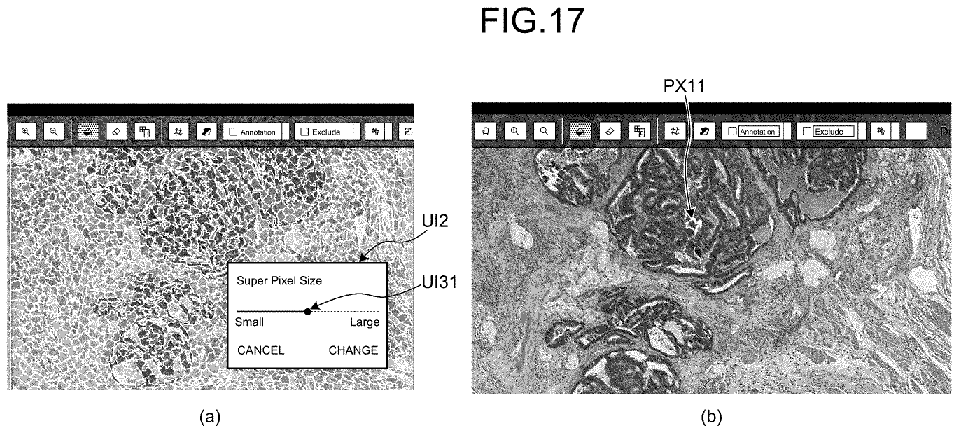

Now, details of display of a super-pixel will be given. The image analyzer 100 visualizes super-pixels in the whole of the display area, for example, for a user to determine the sizes of the super-pixels. Below, visualization in the image analyzer 100 will be described with reference to FIG. 17.

FIG. 17 illustrates examples of a pathological image for explaining visualization in the image analyzer 100. FIG. 17(a) illustrates a pathological image in which super-pixels are visualized in the whole of the display area. Additionally, all of the regions surrounded by the white lines are super-pixels. The user adjusts the sizes of the super-pixels by operating a display menu U131 included in a display menu UI2. For example, when the user moves the display menu U131 rightward, the sizes of the super-pixels increase. Then, the image analyzer 100 visualizes the super-pixels having the adjusted sizes in the whole of the display area.

FIG. 17(b) illustrates a pathological image in which only one super-pixel PX11 is visualized in accordance with the movement of a user's operation. In FIG. 17, when the user performs an operation for selecting a super-pixel, the screen transitions from FIG. 17(a) to the pathological image of FIG. 17(b). The image analyzer 100 visualizes only the super-pixel PX11 responsive to the user's operation, for example, to allow the user to select the super-pixel actually. For example, the image analyzer 100 visualizes only the contour of a region ahead of the mouse pointer of the user. Thus, the image analyzer 100 can improve the visibility of the pathological image.

Further, the image analyzer 100 may display a super-pixel in a light color or a color with transparency to such an extent that the super-pixel on the pathological image can be visually recognized. By setting the color for display of the super-pixel to a light color or a color with transparency, it is possible to improve the visibility of the pathological image.

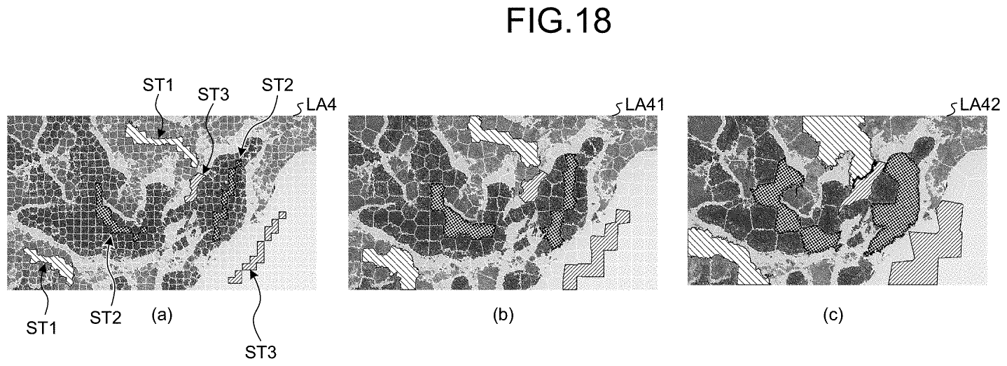

FIG. 18 illustrates examples of a pathological image in which super-pixels are set by segmentation into different numbers of segments. The setting unit 232 sets super-pixels in the pathological image with different numbers of segments in accordance with a user's operation. Adjusting the number of segmentations causes a change in sizes of the super-pixels. FIG. 18(a) illustrates a pathological image in which super-pixels are set by segmentation into the largest number of segments. In this case, the size of each super-pixel is the smallest. FIG. 18(c) illustrates a pathological image in which super-pixels are set by segmentation into the smallest number of segments. In this case, the size of each super-pixel is the largest. The user's operations for designating target regions that are performed on the pathological images of FIGS. 18(b) and 18(c) are the same as the user's operation on the pathological image of FIG. 18(a), and thus, will be described below using FIG. 18(a).

In FIG. 18(a), the target region is designated by the user's selection of super-pixels. Specifically, the range of super-pixels having been designated is the target region. In FIG. 18(a), when the super-pixels are selected, the range of the selected super-pixels are filled in. Additionally, the way of designation is not limited to filling-in the range of super-pixels. For example, the outermost edge of the range of the super-pixels may be indicated by an annotation or the like. Alternatively, the color of the whole of the displayed image may be set to a color (gray or the like, for example) different from that of the original image and only a selected target region is displayed in the color of the original image, thereby improving the visibility of the selected target region. The range of the super-pixels is ST1, ST2, or ST3, for example. The acquisition unit 131 acquires the positional information of the target region designated by the user's selection of the super-pixels via the display device 14, from the display control device 13. In FIG. 18(a), in order to distinguish the target regions from each other, the ranges of the super-pixels are filled in using, for example, pieces of different color information. For example, the ranges of the super-pixels are filled in using pieces of different color information or the like in accordance with the similarity of the feature value of each target region calculated by the calculation unit 233. The provision unit 235 provides information for displaying the ranges of the super-pixels on the display device 14 using pieces of different color information or the like, to the display control device 13. In FIG. 18(a), for example, the range of super-pixels filled in with blue is denoted as “ST1”. In FIG. 18(a), for example, the range of super-pixels filled in with red is denoted as “ST2”. In FIG. 18(a), for example, the range of super-pixels filled in with green is denoted as “ST4”. As a result, even in a case where a plurality of images showing different living bodies of high interest to the user are included in a pathological image, it is possible to search for another target region for each target region. Further, it is possible to show another target regions having been searched for based on respective target regions, in different modes, to the user.

### 3-3. Processing Procedure According to Second Embodiment

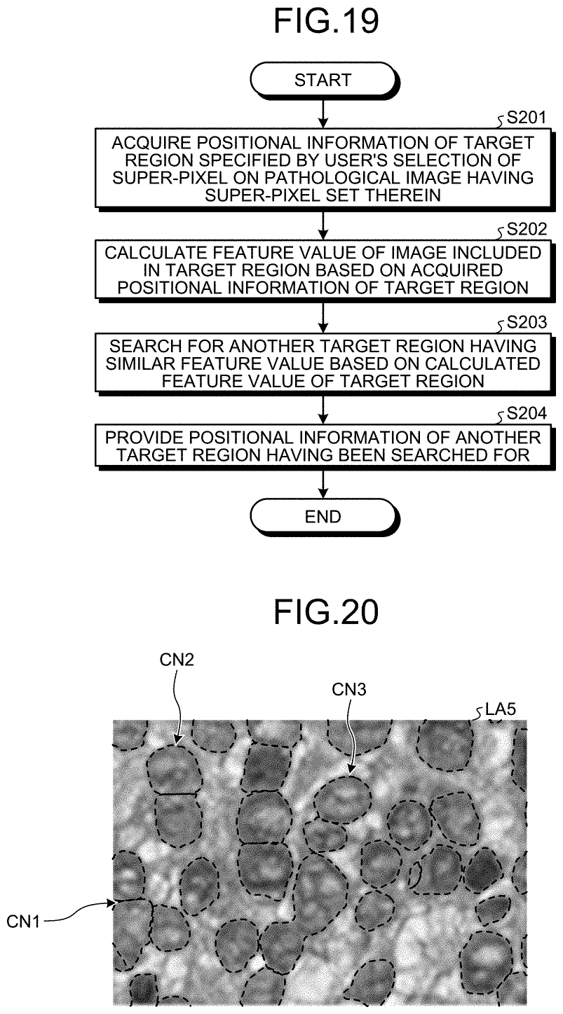

Next, a processing procedure according to the second embodiment will be described with reference to FIG. 19. FIG. 19 is a flowchart illustrating a processing procedure according to the second embodiment. As illustrated in FIG. 19, the image analyzer 200 acquires the positional information of a target region designated by a user's selection of a super-pixel on a pathological image in which the super-pixel is set (step S201). Additionally, the processing after the step S201 is similar to that in the first embodiment, and thus description thereof is omitted.

Hereinabove, in the first embodiment and the second embodiment, the case where the image analyzer 200 searches for another target region that is similar, based on a target region designated by a user on a pathological image has been described. Such processing as performed in the case of searching for another target region similar to the target region based only on information included in a pathological image will be hereinafter referred to as a “normal search mode” if appropriate.

### 4. Modifications of Second Embodiment

The image analysis system 1 according to the above-described second embodiment may be implemented in various different modes other than the above-described embodiment. Then, other implementation modes of the image analysis system 1 will be described below. Description in respects similar to those in the above-described embodiment is omitted.

### 4-1. First Modification: Search Using Cell Information

In the above-described example, the case where a super-pixel is set by segmentation based on the feature value of a pathological image has been described. However, the way of setting a super pixel is not limited to this example. When acquiring a pathological image including an image of a specific living body such as a cell nucleus, the image analyzer 200 may set a super-pixel so as to prevent the region of the image showing the cell nucleus from corresponding to one super-pixel. Below, a specific description will be given.

### 4-1-1. Image Analyzer

The acquisition unit 231 acquires information regarding a pathological image, other than the pathological image. The acquisition unit 231 acquires, as information other than the pathological image, information regarding a cell nucleus that is detected based on the feature value of the pathological image and is included in the pathological image. Meanwhile, a learning model for detecting a cell nucleus, for example, is applied to the detection of the cell nucleus. The learning model for detecting the cell nucleus is generated by learning in which the pathological image is input information and the information regarding the cell nucleus is output information. Further, this learning model is acquired by the acquisition unit 231 via the communication unit 110. The acquisition unit 231 acquires the information regarding the cell nucleus included in the pathological image of interest by inputting the pathological image of interest to the learning model that outputs the information regarding the cell nucleus in response to input of the pathological image. Further, the acquisition unit 231 may acquire a learning model for detecting a specific cell nucleus depending on the type of cell nucleus.

The calculation unit 233 calculates the feature value of the region of the cell nucleus acquired by the acquisition unit 231 and the feature value of a region other than the cell nucleus. The calculation unit 233 calculates the similarity between the feature value of the region of the cell nucleus and the feature value of the region other than the cell nucleus.

The setting unit 232 sets a super-pixel in the pathological image based on the information regarding the cell nucleus acquired by the acquisition unit 231. The setting unit 232 sets a super-pixel based on the similarity calculated by the calculation unit 233, between the feature value of the region of the cell nucleus and the feature value of the region other than the cell nucleus.

### 4-1-2. Information Processing

In FIG. 20, a pathological image includes a plurality of cell nuclei. In FIG. 20, the cell nuclei are indicated by their contours of the dotted lines. Additionally, in FIG. 20, for the purpose of simplifying the illustration, only regions indicating cell nuclei CN1 to CN3 are denoted by reference signs. Though not all the regions indicating the cell nuclei are denoted by reference signs in FIG. 20, actually, each of all the regions having contours indicated by the dotted lines indicates a cell nucleus.

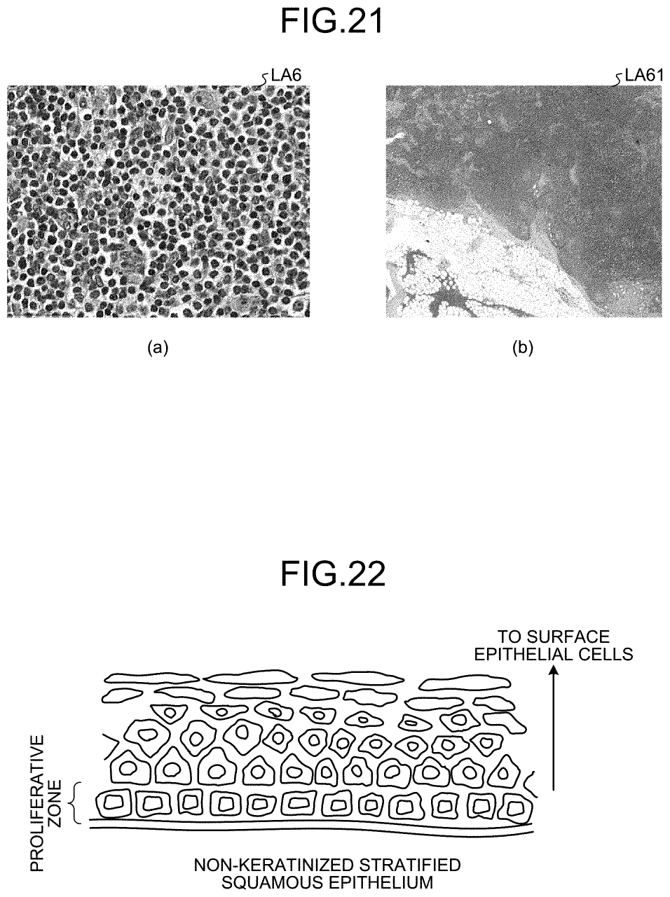

In the meantime, whereas a user can manually designate cell nuclei of a specific type one by one, it takes a huge amount of time and effort. In the case of a high magnification, in particular, due to an infinite number of cells, it is likely that manually designating is difficult. FIG. 21 illustrates how cell nuclei look at different magnifications. FIG. 21(a) illustrates how cell nuclei look at a high magnification. FIG. 21(b) illustrates how cell nuclei look at a low magnification. As illustrated in FIG. 21(b), a user can also determine a target region by inputting a boundary to a pathological image at a low magnification, to set cell nuclei included in the target region as cell nuclei of a specific type that the user desires to designate. However, according to this method, a cell nucleus not desired by the user may be also included in the target region, and thus there is room for further improvement in usability in designation of a cell nucleus of a specific type. Then, in the present embodiment, a plot diagram corresponding to the feature value of a cell nucleus is generated to filter only a specific type of cell nucleus. The feature value of a cell nucleus is, for example, the flatness or the size.

With reference to FIG. 22, the flatness of a cell nucleus will be described. FIG. 22 illustrates an example of the flatness of normal cell nuclei. A stratified squamous epithelium illustrated in FIG. 22 is a non-keratinized stratified squamous epithelium that is a stratified squamous epithelium having cell nuclei also in superficial cells. As illustrated in FIG. 22, in the non-keratinized stratified squamous epithelium, cells are not keratinized. In the drawing, a proliferative zone is an aggregate of cells having proliferative capability for proliferating cells in the surface layer of the epithelium. Then, the cells proliferating from the proliferative zone become more flattened toward the surface layer of the epithelium. In other words, the flatness of the cells proliferating from the proliferative zone increases toward the surface layer. In general, the shape and arrangement including the flatness of cell nuclei are important for pathological diagnosis. It is known that a pathologist or the like performs diagnosis for abnormality of cells based on the flatness of the cells For example, a pathologist or the like diagnoses a cell having a cell nucleus with great flatness in a layer other than the surface layer as a cell that is very likely to be abnormal, based on a distribution indicating the flatness of cells, in some cases. However, it is probably difficult to check the flatness of cell nucleus for performing diagnosis using only information included in an image. Thus, it is desired to perform processing specialized for searching for a cell nucleus included in a pathological image. Such processing as performed in the case of searching for another target region similar to a target region based on information regarding a cell nucleus searched for from a pathological image will be hereinafter referred to as a “cell search mode” as appropriate.

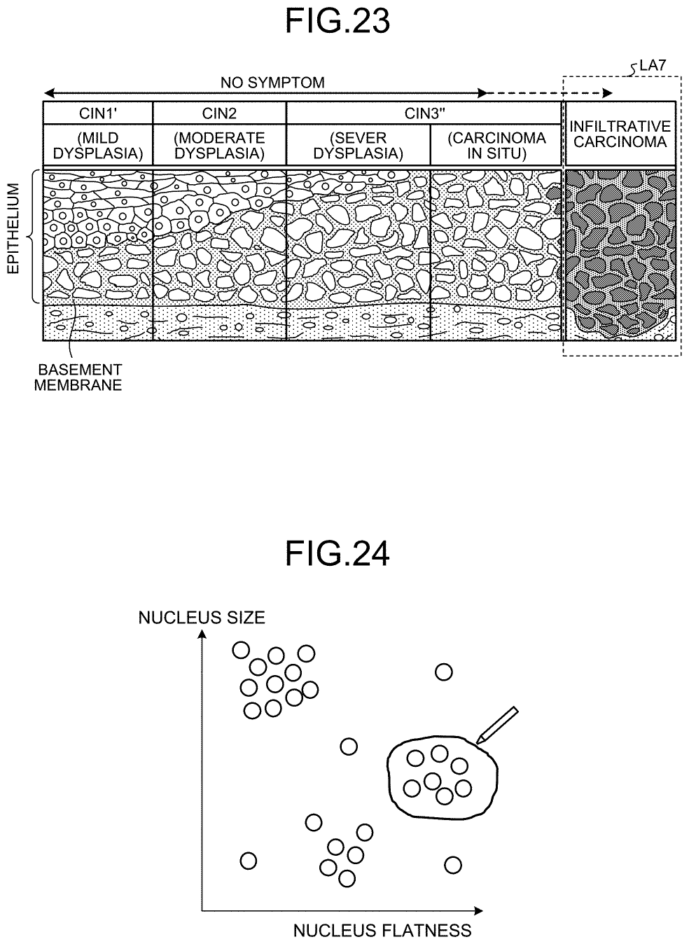

Further, FIG. 23 illustrates an example of the flatness of cell nuclei of abnormal cells. As illustrated in FIG. 23, as the symptom of a lesion advances, cells close to a basement membrane separating a cell layer are keratinized. Specifically, the symptom of a lesion advances from mild to moderate and from moderate to severe, and all the epithelial cells are keratinized by the time when it is diagnosed as cancer. Then, as indicated by “ER1”, if there is a lesion such as a tumor, atypical cells having shapes different from that of a normal cell are distributed everywhere, and break and infiltrate into the basement membrane.

FIG. 24 illustrates a distribution of cell nuclei based on the flatness and sizes of the cell nuclei. In FIG. 24, the vertical axis represents the size of a cell nucleus, and the horizontal axis represents the flatness of a cell nucleus. In FIG. 24, each plot indicates a cell nucleus. In FIG. 24, a user designates a distribution of cell nuclei having specific flatness and specific sizes. In FIG. 24, a distribution included in a range surrounded by the user's freehand line is designated. Additionally, such designation of a distribution by a user's freehand line as illustrated in FIG. 24 is an example, and the way of designation is not limited to this example. For example, a user may designate a specific distribution by surrounding a distribution using a circle, a rectangle or the like having a specific size. In another example, a user may specify some numerical values on the vertical axis and the horizontal axis of a distribution to designate the distribution included in both of ranges on the vertical axis and the horizontal axis based on the numerical values. The distribution based on the flatness and sizes of the cell nuclei is displayed on the display device 14. Upon receipt of the user's designation of the distribution of the cell nuclei displayed on the display device 14, the display control device 13 transmits information regarding the cell nuclei designated on the distribution, to the image analyzer 200. The acquisition unit 231 acquires the information regarding the cell nucleus designated by the user on the distribution. In this manner, the image analyzer 200 may search for another target region using not only the feature value of a super-pixel, but also a plurality of feature values such as the flatness and area of a cell.

### 4-1-3. Processing Procedure

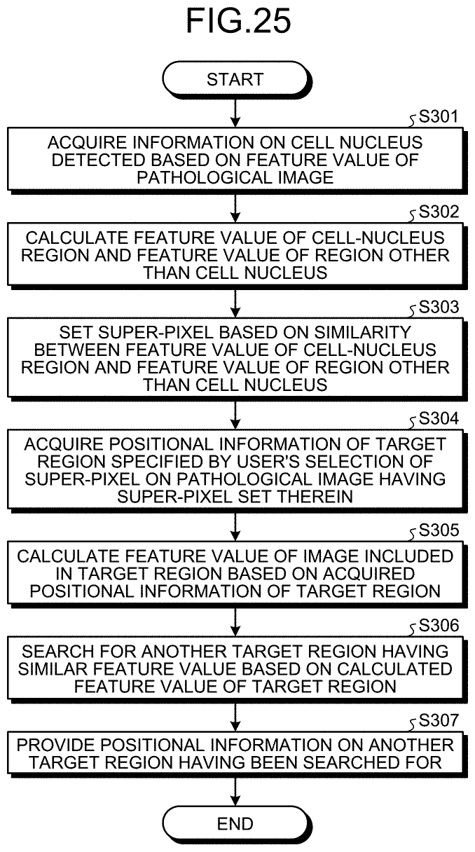

Next, a processing procedure according to the first modification will be described with reference to FIG. 25. FIG. 25 is a flowchart illustrating a processing procedure according to the first modification. As illustrated in FIG. 25, the image analyzer 200 acquires information regarding a cell nucleus detected based on the feature value of a pathological image. Further, the image analyzer 200 calculates the feature value of a region of the cell nucleus and the feature value of a region other than the cell nucleus. The image analyzer 200 sets a super-pixel based on the similarity between the feature value of the region of the cell nucleus and the feature value of the region other than the cell nucleus. Additionally, the processing after the step S304 is similar to that in the second embodiment, and thus description thereof is omitted.

### 4-2. Second Modification: Search Using Organ Information

### 4-2-1. Image Analyzer

In a case where clinical information regarding whole slide imaging of a pathological image of interest can be acquired from a laboratory information system (LIS)) or the like in a hospital, a lesion such as a tumor can be searched for with high accuracy by using the information. For example, it is known that a magnification suitable for search varies depending on the type of tumor. For example, for signet-ring cell carcinoma that is a type of gastric cancer, it is desirable to perform pathological diagnosis at a magnification of about 40 times. It is also possible to automatically set a magnification of an image being searched by acquiring information regarding a lesion such as a tumor from a LIS.

Further, determination of whether a tumor has metastasized greatly affects the future of a patient. For example, if a region similar to a tumor is observed near an infiltration boundary as a result of search of the infiltration boundary based on information regarding an organ, a more appropriate advice can be given to a patient.

Below, processing in which the image analyzer 200 acquires information regarding a target organ in a pathological image to search for another target region will be described. In this case, the image analyzer 200 sets a super-pixel by performing segmentation specialized for a target organ in a pathological image. In general, due to differences in characteristics and size among organs, processing specialized for each organ is desired. Such processing as performed in the case of searching for another target region similar to a target region based on information regarding a target organ in a pathological image will be hereinafter referred to as a “organ search mode” as appropriate. In the organ search mode, the image analyzer 200 further includes a generation unit 236.

The acquisition unit 231 acquires organ information as information other than a pathological image. For example, the acquisition unit 231 acquires organ information regarding an organ specified based on the feature value of the pathological image, such as a stomach, a lung, or a chest. The organ information is acquired, for example, via a LIS. The acquisition unit 231 acquires information for performing segmentation specialized for each organ. For example, in a case where an organ specified based on the feature value of the pathological image is a stomach, the acquisition unit 231 acquires information for performing segmentation of a pathological image of a stomach. The information for performing segmentation specialized for each organ is acquired from, for example, an external information processing apparatus storing therein organ information about each organ.

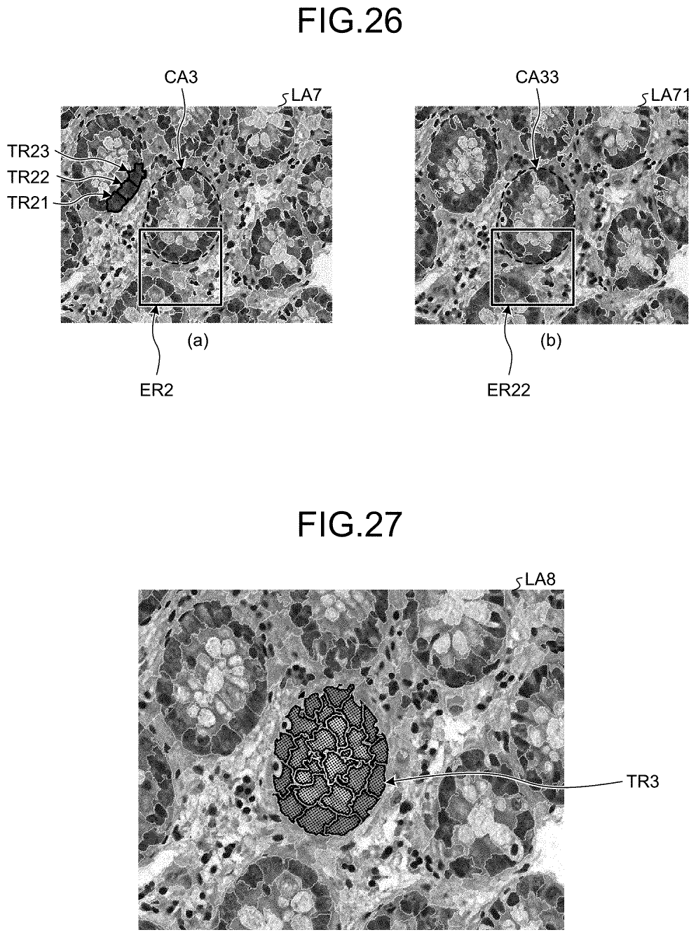

The setting unit 232 sets a super-pixel in the pathological image in accordance with the information that is acquired by the acquisition unit 231 for performing segmentation of the pathological image specialized for each organ. The setting unit 232 sets a super-pixel in the pathological image by performing segmentation specialized for each target organ in the pathological image. In a case where the target organ in the pathological image is a lung, for example, the setting unit 232 sets a super-pixel using a learning model generated by learning of a relationship between the pathological image of the lung and the super-pixel set in the pathological image. Specifically, the setting unit 232 sets a super-pixel in the pathological image of the lung, i.e., the target organ, by inputting the pathological image of the lung, the target organ, to a learning model generated by learning in which the pathological image of the lung having no super-pixel set therein is input information and the super-pixel set in the pathological image is output information. As a result, the setting unit 232 can set a super-pixel with high accuracy for each organ. Below, a success example and a failure example of a super-pixel set by the setting unit 232 will be described with reference to FIGS. 26 to 28.

FIG. 26(a) illustrates a success example of a super-pixel. Additionally, all of the regions surrounded by the white lines are super-pixels. For example, regions TR21 to TR23 surrounded by the dotted lines are examples of a super-pixel. Super-pixels are not limited to the regions TR21 to TR23, and each of all the regions surrounded by the white lines is a super-pixel. As indicated by “ER2”, in the success example of a super-pixel, the setting unit 232 sets super-pixels by performing segmentation for each living body individually. FIG. 26(b) illustrates a failure example of a super-pixel. As indicated by “ER22”, in the failure example of a super-pixel, the setting unit 232 sets super-pixels by performing segmentation so that plural living bodies are intermingled.

FIG. 27 illustrates a target region in a case where super-pixels are successfully set. FIG. 27 illustrates a target region TR3 in a case where a user selects super-pixels including a cell CA3 in “LA7” of FIG. 26(a). As illustrated in FIG. 27, when super-pixels are successfully set, the provision unit 235 can provide the positional information of the target region where plural living bodies are not intermingled, to the display control device 13.

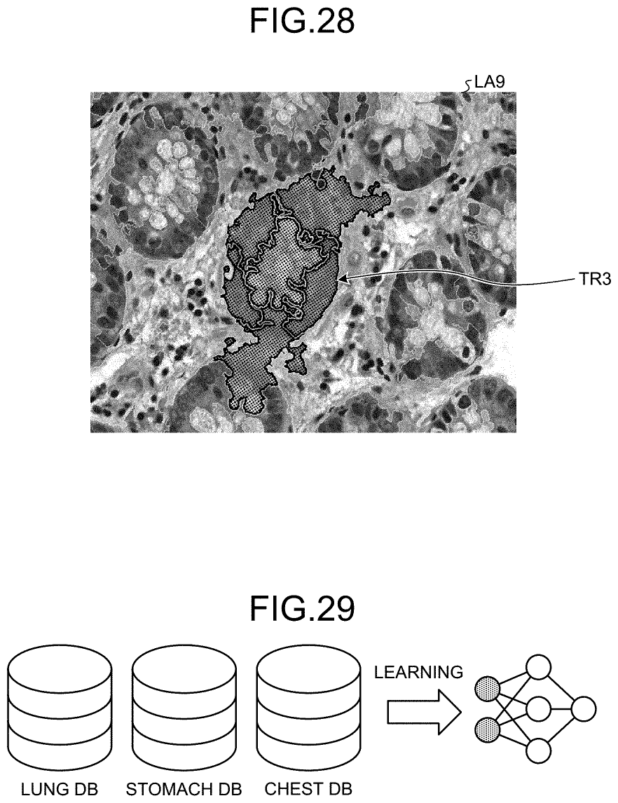

FIG. 28 illustrates a target region in a case where setting of super-pixels ends in failure. FIG. 28 illustrates a target region TR33 in a case where a user selects super-pixels including a cell CA33 in “LA71” of FIG. 26(b). As described above, without segmentation specialized for each organ based on information regarding a target organ in a pathological image, the target region as illustrated in FIG. 28 is probably obtained. Thus, the setting unit 232 can set a super-pixel with high accuracy by performing segmentation specialized for each organ based on information regarding a target organ in a pathological image. Further, in a case where setting of super-pixels ends in failure, the provision unit 235 provides the positional information of the target region where plural living bodies are intermingled, to the display control device 13.

The generation unit 236 generates a learning model for displaying the super-pixels having been subjected to segmentation by the setting unit 232 in a visible state. Specifically, the generation unit 236 generates a learning model for estimating the similarity between images using a combination of the images as input information. Further, the generation unit 236 generates a learning model by learning using a combination of images whose similarity satisfies a predetermined condition, as correct-answer information.

As illustrated in FIG. 29, the acquisition unit 231 acquires a pathological image serving as a material of a combination of images corresponding to correct-answer information from a database of each organ. Then, the generation unit 236 generates a learning model for each organ.

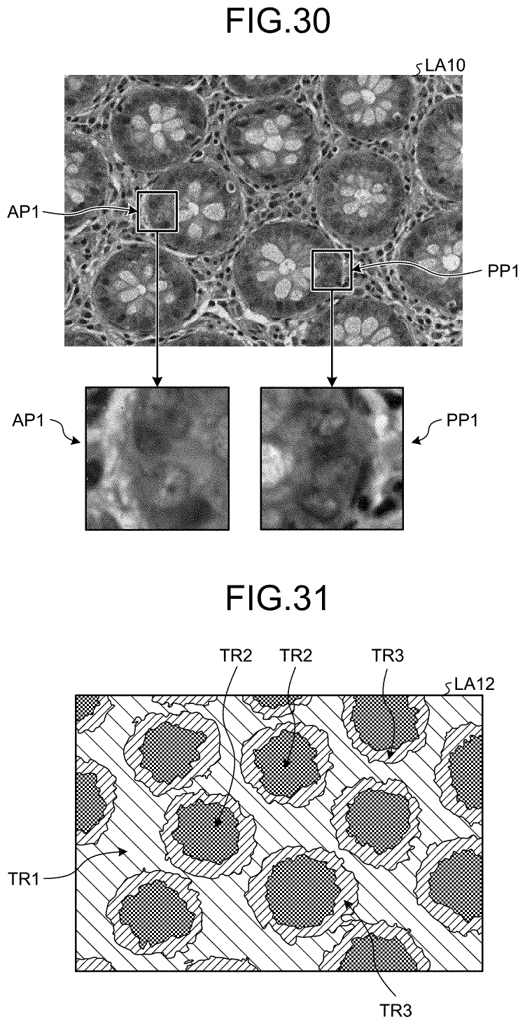

FIG. 30 illustrates a combination of images corresponding to correct-answer information. A region AP1 is a randomly-selected region randomly selected from a pathological image. Further, a region PP1 is a region including an image of a living body similar to that in the region AP1. The region PP1 is a region including an image whose feature value satisfies a predetermined condition. The acquisition unit 231 acquires a combination of the image included in the region AP1 and the image included in the region PP1 as correct-answer information.

Then, the generation unit 236 generates a learning model by learning using the feature value of the image included in the region AP1 and the feature value of the image included in the region PP1 as correct-answer information. Specifically, when a randomly-selected image is input, the image analyzer 100 generates a learning model for estimating the similarity between the image and the image included in the region AP1.

FIG. 31 illustrates an image LA12 in which super-pixels having been subjected to segmentation by the setting unit 232 are displayed in a visible state. In FIG. 31, target regions including images of living bodies having similar feature values are displayed in a visible state. Specifically, the target region TR1, the target region TR2, and a target region TR3 are displayed in a visible state. In this regard, since the target region TR1, the target region TR2, and the target region TR3 indicate images having different feature values, clusters to which the target regions belong are assumed to be different from each other. The generation unit 236 generates a learning model by learning based on training data that is collected so as to include a combination of images randomly acquired from target regions belonging to the same cluster as correct-answer information.

### 4-2-2. Variations of Information Processing

### 4-2-2-1. Acquisition of Correct-Answer Information with Small Amount of Data for Correct-Answer Information

The above-described embodiments has dealt with the case where the generation unit 236 generates a learning model using a combination of images whose feature values satisfy a predetermined condition, as correct-answer information. However, in some cases, the data amount of a combination of images whose feature values satisfy a predetermined condition is insufficient. For example, the data amount of a combination of images whose feature values satisfy a predetermined condition may be insufficient for generating a learning model for estimating similarity with high accuracy. In this case, it is assumed that images close to each other have similar feature values, and a learning model is generated by learning based on training data that is collected using a combination of the images close to each other as correct-answer information.

The acquisition unit 231 acquires an image of a predetermined region included in a pathological image and an image that is located in the vicinity of the predetermined region and has similar feature values such as a color and texture, as a combination of images corresponding to correct-answer information. A generation unit 137 generates a learning model based on this combination of images.

### 4-2-2-2. Learning Using Combination of Images Corresponding to Incorrect-Answer Information

The generation unit 236 may generate a learning model using a combination of images whose feature values do not satisfy a predetermined condition, as incorrect-answer information.

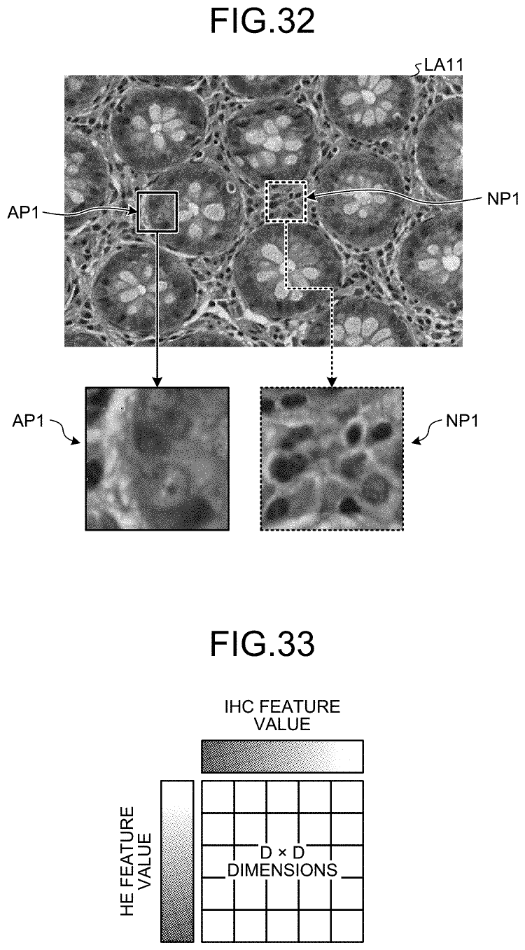

FIG. 32 illustrates a combination of images not corresponding to correct-answer information. A region NP1 is a region including an image of a living body that is not similar to that in the region AP1. Specifically, the region NP1 is a region including an image whose feature value does not satisfy a predetermined condition. The acquisition unit 231 acquires a combination of the image included in the region AP1 and the image included in the region NP1 as incorrect-answer information.

Then, the generation unit 236 generates a learning model by learning in which the feature value of the image included in the region AP1 and the feature value of the image included in the region NP1 correspond to incorrect-answer information.

Further, the generation unit 236 may generate a learning model using correct-answer information and incorrect-answer information. Specifically, the generation unit 236 may generate a learning model by learning in which the image included in the region AP1 and the image included in the region PP1 correspond to correct-answer information and the image included in the region AP1 and the image included in the region NP1 correspond to incorrect-answer information.

### 4-2-2-3. Acquisition of Incorrect-Answer Information with Small Amount of Data for Incorrect-Answer Information

In a case where the data amount of a combination of images corresponding to incorrect-answer information is insufficient, the generation unit 236 may acquire incorrect-answer information based on the following information processing.

The generation unit 236 may acquire an image of a predetermined region included in a pathological image and an image that is not located in the vicinity of the predetermined region and has non-similar feature values such as a color and texture, as a combination of images corresponding to incorrect-answer information.

### 4-3. Third Modification: Search Using Staining Information

### 4-3-1. Image Analyzer

A block piece cut out from a specimen such as an organ of a patient is sliced to prepare a slice. For staining of a slice, various types of staining techniques such as general staining showing the morphology of a tissue, typified by hematoxylin-eosin (HE) staining, or immunostaining showing the immune state of a tissue, typified by immunohistochemistry (IHC) staining, can be applied. In such staining, one slice may be stained using a plurality of different reagents, or two or more slices sequentially cut out from the same block piece (also referred to as adjacent slices) may be stained using different reagents. In general, in some cases, though images of different regions in a pathological image look the same as each other when subjected to general staining, the images of different regions in the pathological image look different from each other when subjected to other staining such as immunostaining. As such, the feature value of an image of a region included in a pathological image varies depending on each staining technique. For example, immunostaining includes staining in which only cell nuclei are stained and staining in which only cell membranes are stained. In searching for another target region based on details of cytoplasm included in a pathological image, HE staining is desired, for example.

Below, search processing of another target region that is performed by the image analyzer 200 and is specialized for a stained pathological image is referred to as a “different staining search mode” as appropriate. In the different staining search mode, another target region is searched for using a plurality of different staining techniques. Additionally, in the different staining search mode, the image analyzer 200 further includes a changing unit 237.

The acquisition unit 231 acquires a plurality of pathological images stained differently.

The setting unit 232 sets a super-pixel in each of the pathological images stained differently, based on the feature value of the pathological image.

The changing unit 237 changes the positional information of the super-pixels so that the images of the living bodies indicated by the respective super-pixels match each other, based on the pieces of positional information of the respective pathological images. For example, the changing unit 237 changes the positional information of the super-pixels based on the features extracted from the images of the living bodies indicated by the respective super-pixels.

The calculation unit 233 calculates the feature value of each of the super-pixels indicating the images of the same living body. Then, the calculation unit 233 aggregates the feature values of the super-pixels indicating the images of the same living body, to calculate a representative feature value. For example, the calculation unit 233 aggregates the feature values of super-pixels having been subjected to different staining techniques, to calculate a representative feature value, that is, a feature value common among the different staining techniques.

FIG. 33 illustrates an example of calculation of a representative feature value. In FIG. 33, the calculation unit 233 calculates a representative feature value based on the feature values of super-pixels having been subjected to HE staining and the feature values of super-pixels having been subjected to IHC staining.

The calculation unit 233 calculates a representative feature value based on vectors indicating the feature values of the super-pixels for the respective staining techniques. In one example of this calculation method, the calculation unit 233 calculates a representative feature value by combining vectors indicating the feature values of the super-pixels for the respective staining techniques. Here, combining vectors means generating a vector including a plurality of vectors in respective dimensions by adding the vectors. For example, the calculation unit 132 calculates the feature value of an eight-dimensional vector as a representative feature value by adding two four-dimensional vectors. In another example, the calculation unit 233 calculates a representative feature value based on the sum, the product, or the linear combination of vectors in each dimension indicating the feature values of the super-pixels for the respective staining techniques. Here, the sum, the product, and the linear combination in each dimension are methods for calculating a representative feature value using the feature values of a plurality of vectors in each dimension. For example, assuming that the feature values of two vectors each in a predetermined dimension are A and B, the calculation unit 132 calculates the sum of A+B, the product of A*B, or the linear sum of W1*A+W2*B for each dimension, thereby calculating a representative feature value. In another different example, the calculation unit 233 calculates a representative feature value based on the direct product of vectors indicating the feature values of the super-pixels for the respective staining techniques. Here, the direct product of vectors is a product of the feature values of a plurality of vectors each in a randomly-selected dimension. For example, the calculation unit 132 calculates a product of the feature values of two vectors each in a randomly-selected dimension, to calculate a representative feature value. For example, in a case where two vectors are four-dimensional vectors, the calculation unit 132 calculates a product of feature values in a randomly-selected dimension, i.e., in four dimensions, to calculate a feature value in a 16-dimensional vector as a representative feature value.

Based on the feature value calculated by the calculation unit 233, the search unit 234 searches for another target region.

The provision unit 235 provides the positional information of another target region searched for in the above-described different staining search mode, to the display control device 13.

### 5. Application of Embodiments

The above-described processing can be applied to various technologies. Below, application examples of the embodiments will be described.

The above-described processing can be applied to generation of annotation data for machine learning. For example, the above-described processing, when applied to a pathological image, can be applied to generation of annotation data for generating information for estimating information regarding pathology of a pathological image. A pathological image is vast and complicated, and thus it is hard to annotate all similar regions in the pathological image. Since the image analyzers 100 and 200 can search for another similar target region that is similar using one annotation, man-power labor can be reduced.

The above-described processing can be applied to extraction of a region that includes tumor cells in the largest amount. In genetic analysis, a region including tumor cells in the largest amount is found and sampled. However, even if a pathologist or the like finds a region including many tumor cells, he cannot confirm whether or not the region includes tumor cells in the largest amount, in some cases. Since the image analyzers 100 and 200 can search for another target region similar to a target region including a lesion found by a pathologist or the like, another lesion can be automatically searched for. The image analyzers 100 and 200 can determine a target region for sampling by specifying the largest target region base on another target region having been searched for.

The above-described processing can be applied to calculation of a quantitative value such as a probability that a tumor is included. In some cases, a probability that a tumor is included is calculated before genetic analysis. To this end, calculation through visual observation by a pathologist or the like may probably increase dispersion. For example, a pathologist or the like requests genetic analysis in a case where the pathologist or the like who has performed pathological diagnosis needs to calculate a probability that a tumor in a slide is included, and also in a case where only visual confirmation by pathology or the like cannot achieve quantitative measurement. The image analyzers 100 and 200 calculate the size of a range of another target region having been searched for, thereby showing the calculated value as a quantitative value to a pathologist or the like.

The above-described processing can be applied to search of a tumor in a rare site. Although automatic search of a tumor by machine learning has been developed, it probably copes with only search of a typical lesion due to a cost for collection of learning data. The image analyzers 100 and 200 can directly search for a tumor by acquiring a target region from past diagnosis data held privately by a pathologist or the like and searching for another target region.

### 6. Other Variations

In the above-described embodiments and modifications, description has been made by using a pathological image as an example of an image of a subject derived from a living body. However, the above-described embodiments and modifications are not limited to processing using a pathological image, and include processing using an image other than a pathological image. For example, in the above-described embodiments and modifications, a “pathological image” may be replaced with a “medical image” for interpretation. Additionally, a medical image may include, for example, an endoscopic image, a magnetic resonance imaging (MRI) image, a computed tomography (CT) image, and the like. In a case where a “pathological image” is replaced with a “medical image”, for interpretation, a “pathologist” and “pathological diagnosis” may be replaced with a “doctor” and “diagnosis”, respectively, for interpretation.

### 7. Hardware Configuration

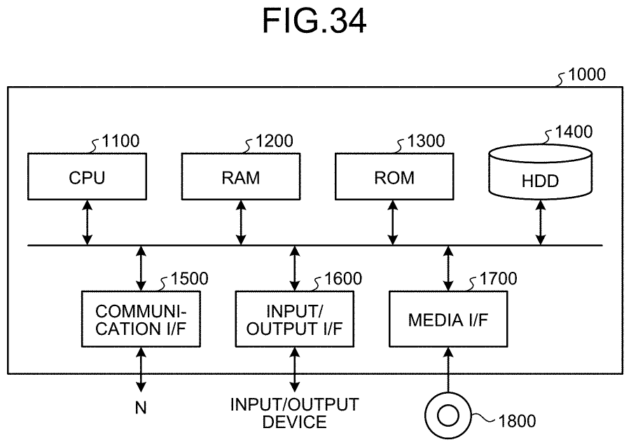

Moreover, the image analyzer 100 or 200 and the terminal system 10 according to the above-described embodiments are realized by a computer 1000 having a configuration as illustrated in FIG. 34, for example. FIG. 34 is a hardware configuration diagram illustrating an example of a computer that implements the functions of the image analyzer 100. The computer 1000 includes a CPU 1100, a RAM 1200, a ROM 1300, a HDD 1400, a communication interface (I/F) 1500, an input/output interface (I/F) 1600, and a media interface (I/F) 1700.

The CPU 1100 operates in accordance with a program stored in the ROM 1300 or the HDD 1400, and controls each unit. The ROM 1300 stores therein a boot program executed by the CPU 1100 at the startup of the computer 1000, a program depending on the hardware of the computer 1000, and the like.

The HDD 1400 stores therein a program executed by the CPU 1100, data used in the program, and the like. The communication interface 1500 receives data from another device via a predetermined communication network, transmits the data to the CPU 1100, and transmits data generated by the CPU 1100 to another device via the predetermined communication network.

The CPU 1100 controls an output device such as a display or a printer and an input device such as a keyboard or a mouse via the input/output interface 1600. The CPU 1100 acquires data from the input device via the input/output interface 1600. Further, the CPU 1100 outputs data generated thereby to the output device via the input/output interface 1600.

The media interface 1700 reads a program or data stored in a recording medium 1800 and provides the program or data to the CPU 1100 via the RAM 1200. The CPU 1100 loads the program from the recording medium 1800 onto the RAM 1200 via the media interface 1700, and executes the loaded program. The recording medium 1800 is, for example, an optical recording medium such as a digital versatile disc (DVD) or a phase change rewritable disk (PD), a magneto-optical recording medium such as a magneto-optical disk (MO), a tape medium, a magnetic recording medium, a semiconductor memory, or the like.

For example, in a case where the computer 1000 functions as the image analyzer 100 or 200 according to the embodiments, the CPU 1100 of the computer 1000 executes a program loaded onto the RAM 1200 to implement the functions of the acquisition unit 131, the calculation unit 132, the search unit 133, and the provision unit 134, or the acquisition unit 231, the setting unit 232, the calculation unit 233, the search unit 234, the provision unit 235, the changing unit 237, and the like. While the CPU 1100 of the computer 1000 reads these programs from the recording medium 1800 and executes them, in another example, these programs may be acquired from another device via a predetermined communication network. Further, the HDD 1400 stores therein the image analysis program according to the present disclosure and the data in the storage unit 120.

### 8. Other

In addition, the whole or a part of the processing that has been described as being automatically performed in the above-described embodiments can be manually performed. Further, the whole or a part of the processing that has been described as being manually performed in the above-described embodiments can be automatically performed by known methods. Besides, the processing procedure, the specific names, and the information including various data and the parameters included in the above description and the drawings can be changed to any specific ones unless otherwise specified. For example, the various types of information illustrated in each of the drawings are not limited to the illustrated information.

Further, the components of each device illustrated in the drawings are only required to have the functions and concepts, and are not necessarily required to be physically configured as illustrated in the drawings. In other words, a specific form of separation and integration of each device is not limited to the illustrated form, and the whole or a part thereof can be separated or integrated functionally or physically in a randomly-selected unit depending on each load, each usage condition, or the like.

Further the above-described embodiments can be combined as appropriate, within a range not causing contradiction in the processing.

Though some of the embodiments of the present application have been described in detail hereinabove with reference to the drawings, these are mere examples, and the present invention can be implemented in other forms subjected to various modifications and improvements based on the knowledge of those skilled in the art, including the aspects described in the section of disclosure of the invention.

In addition, the above-described terms “section”, “module”, and “unit” can be read as “means”, “circuit”, or the like. For example, the acquisition unit can be read as an acquisition means or an acquisition circuit.

Moreover, the present technique can also have the following configurations.

(1)

An image analysis method implemented by one or more computers, comprising:

displaying a first image that is an image of a subject derived from a living body;

acquiring information regarding a first region based on a first annotation added to the first image by a user; and

specifying a similar region similar to the first region from a region different from the first region in the first image, or a second image obtained by image capture of a region including at least a part of a region of the subject subjected to capture of the first image, based on the information regarding the first region, and

displaying a second annotation in a second region corresponding to the similar region in the first image.

(2)

The image analysis method according to (1), further comprising

acquiring the first image in response to a request for an image of the subject at a predetermined magnification from the user, wherein,

the first image is an image having a magnification equal to or higher than the predetermined magnification.

(3)

The image analysis method according to (1) or (2), wherein the first image is an image having resolution different from that of the second image.

(4)

The image analysis method according to (3), wherein the second image is an image having resolution higher than that of the first image.

(5)

The image analysis method according to any one of (1) to (4), wherein the first image is the same image as the second image.

(6)

The image analysis method according to any one of (1) to (5), wherein the second image is an image having resolution selected based on a state of the subject.

(7)

The image analysis method according to any one of (1) to (6), wherein a state of the subject includes a type or a progression stage of a lesion of the subject.

(8)

The image analysis method according to any one of (1) to (7), wherein

the first image is an image generated from a third image having resolution higher than that of the first image, and

the second image is an image generated from the third image having resolution higher than that of the second image.

(9)

The image analysis method according to any one of (1) to (8), wherein the first image and the second image are medical images.

(10)

The image analysis method according to (9), wherein the medical image includes at least one of an endoscopic image, an MRI image, and a CT image.

(11)

The image analysis method according to any one of (1) to (10), wherein the first image and the second image are microscopic images.

(12)

The image analysis method according to (11), wherein the microscopic image includes a pathological image.

(13)

The image analysis method according to any one of (1) to (12), wherein the first region includes a region corresponding to a third annotation generated based on the first annotation.

(14)

The image analysis method according to any one of (1) to (13), wherein the information regarding the first region is one or more feature values of an image of the first region.

(15)

The image analysis method according to any one of (1) to (14), wherein

the similar region is extracted from a predetermined region in the second image, and

the predetermined region is a whole image, a display area, or a region set by the user in the second image.

(16)

The image analysis method according to any one of (1) to (15), further comprising

specifying the similar region based on the information regarding the first region and a first discriminant function.

(17)

The image analysis method according to any one of (1) to (16), further comprising

specifying the similar region based on a first feature value calculated based on the information regarding the first region.

(18)

The image analysis method according to any one of (1) to (17), further comprising

storing the first annotation, the second annotation, and the first image while bringing the first annotation, the second annotation, and the first image into correspondence with each other.

(19)

The image analysis method according to any one of (1) to (18), further comprising

generating one or more partial images based on the first annotation, the second annotation, and the first image.

(20)

The image analysis method according to (19), further comprising

generating a second discriminant function based on at least one of the partial images.

(21)

An image generation method implemented by one or more computers, comprising:

displaying a first image that is an image of a subject derived from a living body;

acquiring information regarding a first region based on a first annotation added to the first image by a user; and

specifying a similar region similar to the first region from a region different from the first region in the first image, or a second image obtained by image capture of a region including at least a part of a region of the subject subjected to capture of the first image, based on the information regarding the first region, and

generating an annotated image in which a second annotation is displayed in a second region corresponding to the similar region in the first image.

(22)

A learning-model generation method implemented by one or more computers, comprising:

displaying a first image that is an image of a subject derived from a living body;

acquiring information regarding a first region based on a first annotation added to the first image by a user; and

specifying a similar region similar to the first region from a region different from the first region in the first image, or a second image obtained by image capture of a region including at least a part of a region of the subject subjected to capture of the first image, based on the information regarding the first region, and

generating a learning model based on an annotated image in which a second annotation is displayed in a second region corresponding to the similar region in the first image.

(23)

An annotation apparatus comprising:

an acquisition unit configured to acquire information regarding a first region based on a first annotation added by a user to a first image that is an image of a subject derived from a living body;

a search unit configured to specify a similar region similar to the first region from a region different from the first region in the first image, or a second image obtained by image capture of a region including at least a part of a region of the subject subjected to capture of the first image, based on the information regarding the first region; and

a control unit configured to add a second annotation to a second region corresponding to the similar region in the first image.

(24)

An annotation program causing a computer to execute:

an acquisition procedure of acquiring information regarding a first region based on a first annotation added by a user to a first image that is an image of a subject derived from a living body;

a search procedure of specifying a similar region similar to the first region from a region different from the first region in the first image, or a second image obtained by image capture of a region including at least a part of a region of the subject subjected to capture of the first image, based on the information regarding the first region; and

a control procedure of adding a second annotation to a second region corresponding to the similar region in the first image.

### REFERENCE SIGNS LIST

1 IMAGE ANALYSIS SYSTEM
10 TERMINAL SYSTEM
11 MICROSCOPE
12 SERVER
13 DISPLAY CONTROL DEVICE
14 DISPLAY DEVICE
100 IMAGE ANALYZER
110 COMMUNICATION UNIT
120 STORAGE UNIT
130 CONTROL UNIT
131 ACQUISITION UNIT
132 CALCULATION UNIT
133 SEARCH UNIT
134 PROVISION UNIT
200 IMAGE ANALYZER
230 CONTROL UNIT
231 ACQUISITION UNIT
232 SETTING UNIT
233 CALCULATION UNIT
234 SEARCH UNIT
235 PROVISION UNIT
236 GENERATION UNIT
237 CHANGING UNIT
N NETWORK

1. An image analysis method implemented by one or more computers, comprising:
displaying a first image that is an image of a subject derived from a living body;
acquiring information regarding a first region based on a first annotation added to the first image by a user; and
specifying a similar region similar to the first region from a region different from the first region in the first image, or a second image obtained by image capture of a region including at least a part of a region of the subject subjected to capture of the first image, based on the information regarding the first region, and
displaying a second annotation in a second region corresponding to the similar region in the first image.

2. The image analysis method according to claim 1, further comprising
acquiring the first image in response to a request for an image of the subject at a predetermined magnification from the user, wherein,
the first image is an image having a magnification equal to or higher than the predetermined magnification.

3. The image analysis method according to claim 1, wherein the first image is an image having resolution different from that of the second image.

4. The image analysis method according to claim 3, wherein the second image is an image having resolution higher than that of the first image.

5. The image analysis method according to claim 1, wherein the first image is the same image as the second image.

6. The image analysis method according to claim 1, wherein the second image is an image having resolution selected based on a state of the subject.

7. The image analysis method according to claim 1, wherein a state of the subject includes a type or a progression stage of a lesion of the subject.

8. The image analysis method according to claim 1, wherein
the first image is an image generated from a third image having resolution higher than that of the first image, and
the second image is an image generated from the third image having resolution higher than that of the second image.

9. The image analysis method according to claim 1, wherein the first image and the second image are medical images.

10. The image analysis method according to claim 9, wherein the medical image includes at least one of an endoscopic image, an MRI image, and a CT image.

11. The image analysis method according to claim 1, wherein the first image and the second image are microscopic images.

12. The image analysis method according to claim 11, wherein the microscopic image includes a pathological image.

13. The image analysis method according to claim 1, wherein the first region includes a region corresponding to a third annotation generated based on the first annotation.

14. The image analysis method according to claim 1, wherein the information regarding the first region is one or more feature values of an image of the first region.

15. The image analysis method according to claim 1, wherein
the similar region is extracted from a predetermined region in the second image, and
the predetermined region is a whole image, a display area, or a region set by the user in the second image.

16. The image analysis method according to claim 1, further comprising
specifying the similar region based on the information regarding the first region and a first discriminant function.

17. The image analysis method according to claim 1, further comprising
specifying the similar region based on a first feature value calculated based on the information regarding the first region.

18. The image analysis method according to claim 1, further comprising
storing the first annotation, the second annotation, and the first image while bringing the first annotation, the second annotation, and the first image into correspondence with each other.

19. The image analysis method according to claim 1, further comprising
generating one or more partial images based on the first annotation, the second annotation, and the first image.

20. The image analysis method according to claim 19, further comprising
generating a second discriminant function based on at least one of the partial images.

21. An image generation method implemented by one or more computers, comprising:
displaying a first image that is an image of a subject derived from a living body;
acquiring information regarding a first region based on a first annotation added to the first image by a user; and
specifying a similar region similar to the first region from a region different from the first region in the first image, or a second image obtained by image capture of a region including at least a part of a region of the subject subjected to capture of the first image, based on the information regarding the first region, and
generating an annotated image in which a second annotation is displayed in a second region corresponding to the similar region in the first image.

22. A learning-model generation method implemented by one or more computers, comprising:
displaying a first image that is an image of a subject derived from a living body;
acquiring information regarding a first region based on a first annotation added to the first image by a user; and
specifying a similar region similar to the first region from a region different from the first region in the first image, or a second image obtained by image capture of a region including at least a part of a region of the subject subjected to capture of the first image, based on the information regarding the first region, and
generating a learning model based on an annotated image in which a second annotation is displayed in a second region corresponding to the similar region in the first image.

23. An annotation apparatus comprising:
an acquisition unit configured to acquire information regarding a first region based on a first annotation added by a user to a first image that is an image of a subject derived from a living body;
a search unit configured to specify a similar region similar to the first region from a region different from the first region in the first image, or a second image obtained by image capture of a region including at least a part of a region of the subject subjected to capture of the first image, based on the information regarding the first region; and
a control unit configured to add a second annotation to a second region corresponding to the similar region in the first image.

24. An annotation program causing a computer to execute:
an acquisition procedure of acquiring information regarding a first region based on a first annotation added by a user to a first image that is an image of a subject derived from a living body;
a search procedure of specifying a similar region similar to the first region from a region different from the first region in the first image, or a second image obtained by image capture of a region including at least a part of a region of the subject subjected to capture of the first image, based on the information regarding the first region; and
a control procedure of adding a second annotation to a second region corresponding to the similar region in the first image.
